(19) United States
(12) Patent Application Publication
(10) Pub. No.: US 2022/0087962 A1
(43) Pub. Date: Mar. 24, 2022

(54) COMPOSITIONS AND METHODS FOR TREATING AGE-RELATED DISEASES OR CONDITIONS

(71) Applicant: Arizona Board of Regents on Behalf of the University of Arizona, Tucson, AZ (US)

(72) Inventor: George L. Sutphin, Tucson, AZ (US)

(21) Appl. No.: 17/426,416

(22) PCT Filed: Jan. 30, 2020
(86) PCT No.: PCT/US2020/015855

§ 371 Date: Jul. 28, 2021

Related U.S. Application Data
(60) Provisional application No. 62/798,708, filed on Jan. 30, 2019.

(51) Int. Cl. A61K 31/196; A61K 31/713; A61P 39/06; G01N 33/573

(52) U.S. Cl. CPC A61K 31/196; A61K 31/713; G01N 2333/90241; G01N 33/573; A61P 39/06

(57) ABSTRACT

The disclosure relates to methods of preventing or treating age-related diseases or conditions in a subject. The method comprises targeting the kynurenine pathway.

### CROSS REFERENCE TO RELATED APPLICATIONS

This application claims the benefit of U.S. Provisional Application No. 62/798,708, filed on Jan. 30, 2019. The entire content of which is hereby incorporated herein by reference.

### BACKGROUND

The proportion of Americans over age 65 is expected to increase from 15% in 2019 to more than 24% by 2060. Aging is accompanied by decreased health, increased frailty, and increased incidence of disease. Among the top 10 causes of death in America, 6 share age as the primary risk factor. Combined, these facts place a substantial and growing health and economic burden on society. Interventions that slow the underlying molecular processes that drive aging have the potential to delay functional deterioration with age, extend the healthy portion of the human lifespan, and delay or treat multiple categories of age-associated disease simultaneously. Of particular note is Alzheimer's disease (AD), the sixth leading cause of the death in the United States and the most common form of neurodegeneration in the elderly. The few drugs currently approved to treat AD target cholinergic and glutamatergic signaling in neurons, improving symptoms without affecting the underlying pathology. Hundreds of clinical trials targeting different aspects of AD neuropathology—amyloid toxicity, tau toxicity, oxidative stress, neuroinflammation—have failed to demonstrate efficacy. Identifying effective treatments for AD is a major current focus of scientific resources, and there is a need for novel therapeutic strategies.

### SUMMARY

Disclosed herein are methods of preventing or reducing oxidative stress in a subject, the method comprising administering to the subject a therapeutically effective amount of a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby preventing or reducing oxidative stress in the subject.

Disclosed herein are methods of increasing 3-hydroxyanthranilic acid (3HAA) in a subject, the method comprising administering to the subject a composition that: a) reduces expression of 3-hydroxyanthranilic acid dioxygenase (HAAO); b) reduces expression of HAAO; c) inhibits HAAO activity d) increases the bioavailability 3-hydroxyanthranilic acid (3HAA); e) directly supplements 3HAA; or f) two or more of a) to e) in combination, thereby increasing 3HAA in the subject.

Disclosed herein are methods of decreasing quinolinic acid (QA) in a subject, the method comprising administering to the subject a composition that: a) reduces expression of 3-hydroxyanthranilic acid dioxygenase (HAAO); b) reduces expression of HAAO; or c) inhibits HAAO activity; d) degrades QA; e) increases expression of quinolinic acid phosphoribosyltransferase (QPRT); f) increases expression of QPRT; g) activates QPRT; h) two or more of a) to g) in combination, thereby decreasing QA in the subject.

Disclosed herein are methods of increasing hydrogen peroxide (H₂O₂) breakdown in a subject, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby increasing hydrogen peroxide (H₂O₂) breakdown in a subject.

Disclosed herein are methods of preventing or reducing oxidative stress in a subject, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby preventing or reducing oxidative stress in the subject.

Disclosed herein are methods of preventing one or more proteins from misfolding or aggregating in a subject, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby preventing one or more proteins from misfolding or aggregating in a subject.

Disclosed herein are methods of promoting proper folding of one or more proteins in a subject, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby promoting proper folding of one or more proteins in a subject.

Disclosed herein are methods of increasing or extending a subject's lifespan, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby increasing or extending a subject's lifespan.

Disclosed herein are methods of delaying or preventing the decline in a subject's physical health with age, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby delaying or preventing the decline in a subject's physical health with age.

Disclosed herein are methods of delaying paralysis in a subject expressing amyloid-beta in body wall muscle, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby delaying paralysis in a subject expressing amyloid-beta in body wall muscle.

Disclosed herein are methods of delaying or preventing cognitive decline in a subject with Alzheimer's disease, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby delaying or preventing cognitive decline in a subject with Alzheimer's disease.

Disclosed herein are methods of delaying or preventing cognitive decline in a subject with Huntington's disease, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby delaying or preventing cognitive decline in a subject with Huntington's disease.

Disclosed herein are methods of preventing or reducing neurofibrillary tangles in a subject, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby preventing or reducing neurofibrillary tangles in a subject.

Disclosed herein are methods of treating a disease or a condition associated with misfolding of a protein or protein aggregation in a subject, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby treating a disease or a condition associated with misfolding of a protein or protein aggregation in a subject.

Disclosed herein are methods of reducing amyloid-beta production, misfolding, or aggregation in a subject, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby amyloid-beta production, misfolding, or aggregation in a subject.

Disclosed herein are methods of reducing inflammation in a subject, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby reducing inflammation in a subject.

Disclosed herein are methods of preventing or reducing oxidative stress in a subject in a subject, the method comprising administering to the subject in need thereof a therapeutically effective amount of a composition that a) increases production, concentration, or activity of 3HAA; b) decreases production, concentration, or activity of QA; c) degrades or increases degradation of H₂O₂; or d) two or more of a) to c) in combination.

Disclosed herein are methods of preventing one or more proteins from misfolding or aggregating in a subject, the method comprising administering to the subject in need thereof a therapeutically effective amount of a composition that a) increases production, concentration, or activity of 3HAA; b) decreases production, concentration, or activity of QA; c) degrades or increases degradation of H₂O₂; or d) two or more of a) to c) in combination.

Disclosed herein are methods of increasing or extending a subject's lifespan, the method comprising administering to the subject in need thereof a therapeutically effective amount of a composition that a) increases production, concentration, or activity of 3HAA; b) decreases production, concentration, or activity of QA; c) degrades or increases degradation of H₂O₂; or d) two or more of a) to c) in combination.

Disclosed herein are methods of delaying or preventing the decline in a subject's physical health with age, the method comprising administering to the subject in need thereof a therapeutically effective amount of a composition that a) increases production, concentration, or activity of 3HAA; b) decreases production, concentration, or activity of QA; c) degrades or increases degradation of H₂O₂; or d) two or more of a) to c) in combination.

Disclosed herein are methods of delaying paralysis in a subject expressing amyloid-beta in body wall muscle, the method comprising administering to the subject in need thereof a therapeutically effective amount of a composition that a) increases production, concentration, or activity of 3HAA; b) decreases production, concentration, or activity of QA; c) degrades or increases degradation of H₂O₂; or d) two or more of a) to c) in combination.

Disclosed herein are methods of delaying or preventing cognitive decline in a subject with Alzheimer's disease, the method comprising administering to the subject in need thereof a therapeutically effective amount of a composition that a) increases production, concentration, or activity of 3HAA; b) decreases production, concentration, or activity of QA; c) degrades or increases degradation of H₂O₂; or d) two or more of a) to c) in combination.

Disclosed herein are methods of delaying or preventing cognitive decline in a subject with Huntington's disease, the method comprising administering to the subject in need thereof a therapeutically effective amount of a composition that a) increases production, concentration, or activity of 3HAA; b) decreases production, concentration, or activity of QA; c) degrades or increases degradation of H₂O₂; or d) two or more of a) to c) in combination.

Disclosed herein are methods of preventing or reducing neurofibrillary tangles in a subject, the method comprising administering to the subject in need thereof a therapeutically effective amount of a composition that a) increases production, concentration, or activity of 3HAA; b) decreases production, concentration, or activity of QA; c) degrades or increases degradation of H₂O₂; or d) two or more of a) to c) in combination.

Disclosed herein are methods of treating a disease or a condition associated with misfolding of a protein or protein aggregation in a subject, the method comprising administering to the subject in need thereof a therapeutically effective amount of a composition that a) increases production, concentration, or activity of 3HAA; b) decreases production, concentration, or activity of QA; c) degrades or increases degradation of H₂O₂; or d) two or more of a) to c) in combination.

Disclosed herein are methods of reducing amyloid-beta production, misfolding, or aggregation in a subject, the method comprising administering to the subject in need thereof a therapeutically effective amount of a composition that a) increases production, concentration, or activity of 3HAA; b) decreases production, concentration, or activity of QA; c) degrades or increases degradation of H₂O₂; or d) two or more of a) to c) in combination.

Disclosed herein are methods of reducing inflammation in a subject, the method comprising administering to the subject in need thereof a therapeutically effective amount of a composition that a) increases production, concentration, or activity of 3HAA; b) decreases production, concentration, or activity of QA; c) degrades or increases degradation of H₂O₂; or d) two or more of a) to c) in combination.

Disclosed herein are methods of preventing one or more proteins from misfolding or aggregating, the method comprising administering a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby preventing one or more proteins from misfolding or protein aggregation.

Disclosed herein are methods of promoting proper folding of one or more proteins, the method comprising administering a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby promoting proper folding of one or more proteins.

Disclosed herein are methods of increasing H₂O₂ break down, the method comprising administering a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby increasing H₂O₂ degradation.

Disclosed herein are methods of increasing or extending a subject's lifespan, the method comprising administering a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby increasing or extending the subject's lifespan.

Disclosed herein are methods of delaying paralysis in a subject expressing amyloid-beta in body wall muscle, the method comprising administering to the subject a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby delaying paralysis in the subject.

Disclosed herein are methods of delaying paralysis in a subject with Huntington's disease, the method comprising administering to the subject a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby delaying paralysis in the subject.

Disclosed herein are methods of decreasing quinolinic acid in a subject, the method comprising administering to the subject a composition that: a) reduces expression of 3-hydroxyanthranilic acid dioxygenase (HAAO); b) reduces expression of HAAO; or c) inhibits HAAO activity; thereby decreasing quinolinic acid in the subject.

Disclosed herein are methods of preventing or reducing neurofibrillary tangles in a subject, the method comprising administering to the subject a composition that: a) reduces expression of 3-hydroxyanthranilic acid dioxygenase (HAAO); b) reduces expression of HAAO; c) inhibits HAAO activity; d) decrease the concentration of quinolinic acid (QA); e) increases the bioavailability 3-hydroxyanthranilic acid (3HAA); f) increases the concentration of 3-hydroxyanthranilic acid (3HAA); or g) one or more of a) to c) in combination with d), e) and/or f), thereby preventing or reducing neurofibrillary tangles in the subject.

Disclosed herein are methods of preventing protein misfolding or protein aggregation in a subject, the method comprising administering to the subject in need thereof a therapeutically effective amount of a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby preventing protein misfolding or protein aggregation in the subject.

Disclosed herein are methods of treating a disease or a condition associated with misfolding of a protein or protein aggregation in a subject, the method comprising administering to the subject in need thereof a therapeutically effective amount of a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby treating a disease or a condition associated with misfolding of a protein or protein aggregation in the subject.

Disclosed herein are methods of reducing amyloid-beta production in a subject, the method comprising administering to the subject in need thereof a therapeutically effective amount of a composition that degrades H₂O₂ or increases degradation of H₂O₂.

Disclosed herein are methods of preventing oxidative stress or increasing H₂O₂ degradation in a subject, the method comprising administering to the subject in need thereof a therapeutically effective amount of a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby preventing oxidative stress or increasing H₂O₂ degradation in the subject.

Disclosed herein are methods of reducing inflammation in a subject, the method comprising administering to the subject in need thereof a therapeutically effective amount of a composition that reduces production of, concentration of, or activity of quinolinic acid (QA).

Disclosed herein are methods of reducing inflammation in a subject, the method comprising administering to the subject in need thereof a therapeutically effective amount of a composition that reduces production of, concentration of, or activity of 3-hydroxyanthranilic acid (3HAA).

Disclosed herein are methods of reducing or preventing neurofibrillary tangle formation, the method comprising administering to a subject in need thereof a therapeutically effective amount of a composition that reduces production of, concentration of, or activity of quinolinic acid (QA).

Disclosed herein are methods of screening for an agent or combination of agents effective in preventing or reducing oxidative stress; preventing one or more proteins from misfolding or aggregating; promoting proper folding of one or more proteins; increasing H₂O₂ breakdown; increasing or extending a subject's lifespan; decreasing quinolinic acid; preventing or reducing neurofibrillary tangles; preventing protein misfolding or protein aggregation; treating a disease or a condition associated with misfolding of a protein or protein aggregation or a symptom thereof; reducing amyloid-beta production; preventing oxidative stress or increasing H₂O₂ degradation; reducing inflammation; and/or reducing or preventing neurofibrillary tangle formation comprising: a) determining the expression level of 3-hydroxyanthranilic acid dioxygenase (HAAO) and/or HAAO in a sample from the subject; b) contacting the sample with the an agent or combination of agents; c) determining the expression level of the HAAO and/or HAAO in the sample from the subject; and d) comparing the expression level to the expression level of HAAO or HAAO, respectively in a sample from the subject prior to contacting the sample with the agent or combination of agents to the expression level of HAAO or HAAO in the sample after contacting the sample with the agent or combination of agents, wherein an increase in the expression level or an increase in the activity of the HAAO and/or HAAO in the subject after contacting the sample with the agent or combination of agents indicates efficacy of the agent or combination of agents.

Disclosed herein are methods of screening for an agent or combination of agents thereof effective in preventing or reducing oxidative stress; preventing one or more proteins from misfolding or aggregating; promoting proper folding of one or more proteins; increasing H₂O₂ breakdown; increasing or extending a subject's lifespan; decreasing quinolinic acid; preventing or reducing neurofibrillary tangles; preventing protein misfolding or protein aggregation; treating a disease or a condition associated with misfolding of a protein or protein aggregation or a symptom thereof; reducing amyloid-beta production; preventing oxidative stress or increasing H₂O₂ degradation; reducing inflammation; and/or reducing or preventing neurofibrillary tangle formation comprising: a) determining the activity level of HAAO and/or HAAO in a sample from the subject; b) contacting the sample with the agent or combination of agents; c) determining the activity level of the HAAO and/or HAAO in the sample from the subject; and d) comparing the activity level to the activity level of HAAO and/or HAAO in a sample from the subject prior to contacting the sample with the agent or combination of agents to the activity level of HAAO or HAAO in the sample after contacting the sample with the agent or combination of agents, wherein an increase in the activity of the HAAO and/or HAAO in the subject after contacting the sample with the agent or combination of agents indicates efficacy of the agent or combination of agents.

Disclosed herein are methods of screening for an agent or combination of agents effective in ameliorating one or more symptoms associated with an aberrant kynurenine metabolism associated condition comprising: a) determining the expression level of HAAO and/or HAAO in a sample; b) contacting the agent or combination of agents with the sample; c) determining the expression level of the HAAO and/or HAAO in the sample; and d) comparing the expression level to the expression level of HAAO and/or HAAO prior to contacting the sample with the agent or combination of agents to the expression level of the HAAO and/or HAAO in the sample after contacting the sample with the agent or combination of agents, wherein an increase in the expression level of the HAAO and/or HAAO in the sample after administration indicates efficacy of the agent or combination of agents.

Disclosed herein are methods of screening for an agent or combination of agents effective in treating an aberrant kynurenine metabolism associated condition comprising: a) determining the activity of HAAO and/or HAAO in a sample; b) contacting the sample with the agent or combination of agents; c) determining the activity of HAAO and/or HAAO in the sample from the subject; and d) comparing the activity level to the activity level of HAAO and/or HAAO prior to contacting the sample with the agent or combination of agents, wherein an increase in the activity of HAAO and/or HAAO in the subject after administration indicates efficacy of the agent or combination of agents.

Other features and advantages of the present compositions and methods are illustrated in the description below, the drawings, and the claims.

### BRIEF DESCRIPTION OF THE DRAWINGS

FIG. 1 shows tryptophan metabolism through the kynurenine pathway.

FIG. 2 is an illustration of a mechanistic model linking HAAO, 3HAA, QA, and H₂O₂ to amyloid and tau pathology during AD.

FIGS. 3A-D shows that 3HAA mediates lifespan extension by haao-1 knockdown. FIG. 3A shows that knockdown of haao-1, kynu-1, or tdo-2 increases lifespan in C. elegans. FIG. 3B shows that haao-1(RNAi) increases physiological 3HAA. FIG. 3C shows that 3HAA supplementation extends lifespan, mimicking haao-1 knockdown (D).

FIGS. 4A-B show that 3HAA or haao-1 (RNAi) delays paralysis in C. elegans expressing human Aβ₁₋₄₂ (modeling Alzheimer's disease; FIG. 4A) or a 35-unit polyglutamine repeat peptide (modeling Huntington's disease; FIG. 4B) in body wall muscle.

FIGS. 5A-D show that 3HAA improves oxidative stress resistance and directly degrades H₂O₂. FIG. 5A shows that haao-1 knockdown or 3HAA supplementation increases resistance to paraquat (PQ). FIG. 5B shows that H₂O₂ visibly reduces 3HAA in media. FIG. 5C shows that 3HAA directly degrades H₂O₂. FIG. 5D shows that haao-1 knockout or 3HAA supplementation reduces H₂O₂ produced by C. elegans in vivo.

FIG. 6 shows the stress, stress response pathways, and disease states form a complex network.

FIG. 7 shows stress response pathways in C. elegans.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, worm, etc.). In one aspect, a subject is a mammal. In another aspect, the subject is a human. The term does not denote a particular age or sex. Thus, adult, child, adolescent and newborn subjects, as well as fetuses, whether male or female, are intended to be covered. As used herein, a “subject” is the same as a “patient,” and the terms can be used interchangeably.

As used herein, the term “patient” refers to a subject afflicted with a disease, disorder or condition. The term “patient” includes human and veterinary subjects. In some aspects of the disclosed methods, the “patient” has been diagnosed with a need for treatment for preventing or treating, such as, for example, prior to the administering step.

As used herein, “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

As used herein, the term “reference,” “reference expression,” “reference sample,” “reference value,” “control,” “control sample” and the like, when used in the context of a sample or expression level of one or more genes or proteins refers to a reference standard wherein the reference is expressed at a constant level among different (i.e., not the same tissue, but multiple tissues) tissues, and is unaffected by the experimental conditions, and is indicative of the level in a sample of a predetermined disease status (e.g., not suffering from Alzheimer's disease). The reference value can be a predetermined standard value or a range of predetermined standard values, representing no illness, or a predetermined type or severity of illness.

Determining the expression level of one or more genes or proteins or the activity level of one or more genes or proteins disclosed herein can include determining whether the gene or protein is upregulated or increased as compared to a control or reference sample, downregulated or decreased compared to a control or reference sample, or unchanged compared to a control or reference sample. As used herein, the terms, “upregulated” and “increased expression level” or “increased level of expression” refers to a sequence corresponding to one or more genes disclosed herein that is expressed wherein the measure of the quantity of the sequence exhibits an increased level of expression when compared to a reference sample or “normal” control. For example, the terms, “upregulated” and “increased expression level” or “increased level of expression” refers to a sequence corresponding to one or more genes disclosed herein that is expressed wherein the measure of the quantity of the sequence exhibits an increased level of expression of one or more protein(s) and/or mRNA when compared to the expression of the same mRNA(s) from a reference sample or “normal” control. An “increased expression level” refers to an increase in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more. As used herein, the terms “downregulated,” “decreased level of expression,” or “decreased expression level” refers to a sequence corresponding to one or more genes or proteins disclosed herein that is expressed wherein the measure of the quantity of the sequence exhibits a decreased level of expression when compared to a reference sample or “normal” control For example, the terms “downregulated,” “decreased level of expression,” or “decreased expression level” refers to a sequence corresponding to one or more genes or proteins disclosed herein that is expressed wherein the measure of the quantity of the sequence exhibits a decreased level of expression of one or more protein(s) and/or mRNA when compared to the expression of the same mRNA(s) from a reference sample or “normal” control. A “decreased level of expression” refers to a decrease in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more. Similarly, measuring the activity level, changes in bioavailability of one or more proteins or changes in the concentration of one or more proteins can be compared to the activity level amount available or the concentration of the one or more proteins of the same protein from a reference sample or “normal” control. Decreases in activity, bioavailability or concentration, for example, can be at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more. Increases in activity, bioavailability or concentration, for example, can be at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. In some aspects of the disclosed methods, the subject has been diagnosed with a need for treatment of a viral infection prior to the administering step. As used herein, the phrase “identified to be in need of treatment for a disorder,” or the like, refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

The terms “administering” and “administration” refer to any method of providing a pharmaceutical preparation to a subject. Such methods are well known to those skilled in the art and include, but are not limited to, oral administration, sublingual administration, trans-buccal mucosa administration, transdermal administration, administration by inhalation, nasal administration, topical administration, intravaginal administration, ophthalmic administration, intraaural administration, intracerebral administration, intrathecal administration, rectal administration, intraperitoneal administration, and parenteral administration, including injectable such as intravenous administration, intra-arterial administration, intramuscular administration, intradermal administration, and subcutaneous administration. Ophthalmic administration can include topical administration, subconjunctival administration, sub-Tenon's administration, epibulbar administration, retrobulbar administration, intra-orbital administration, and intraocular administration, which includes intra-vitreal administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “therapeutically effective amount” refers to an amount that is sufficient to achieve the desired result or to have an effect on an undesired condition.

Alzheimer's Disease

Most clinical trials for Alzheimer's disease (AD) involve monotherapies, drugs designed to target a single form of pathology. Limiting production or increasing clearance of Aβ is the most common target. Disappointing clinical results suggest that intervening in a single pathology may be insufficient to delay disease progression. A recent strategy has emerged to target multiple forms of pathology simultaneously (Robinson J L, et al. Brain J Neurol 2018, 141:2181-93; Kametani F, Hasegawa M. Front Neurosci 2018, 12; and Sahoo A K, et al. J Ethnopharmacol 2018, 215:42-73). Described herein are multi-target interventions for AD.

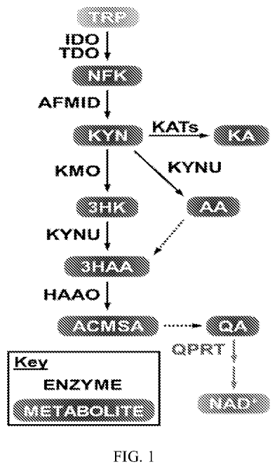

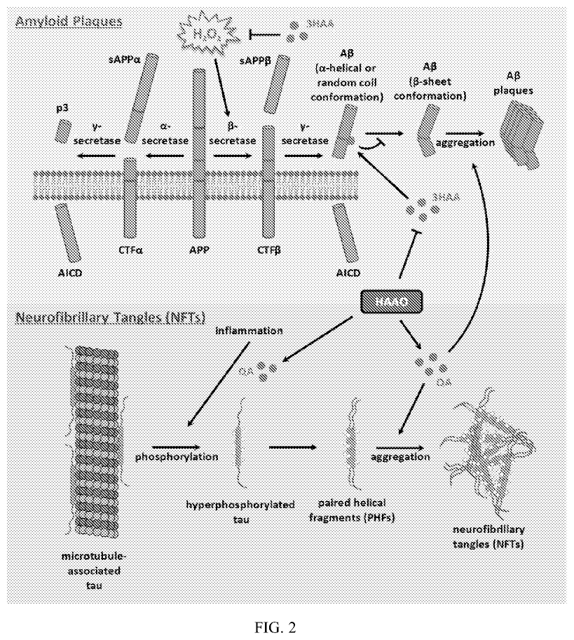

Also disclosed herein are methods of targeting and inhibiting the kynurenine pathway enzyme 3-hydroxyanthranilic acid dioxygenase (HAAO) to simultaneously improve amyloid and tau pathology in AD. The kynurenine pathway metabolizes tryptophan (TRP) through one of two branches, producing either kynurenic acid (KA) or nicotinamide adenine dinucleotide (NAD⁺) (FIG. 1). HAAO catalyzes the conversion of 3HAA to QA in the NAD⁺ branch. Reducing HAAO activity elevates physiological 3HAA and reduces QA, both systemically and in the brain (Parrott J M, et al. Transl Psychiatry 2016; 6:e918-e918; Parli C J, et al. Arch Biochem Biophys 1980, 203:161-166, Fornstedt-Wallin B, et al. Eur J Pharmacol 1999, 386:15-24; Linderberg M, et al. Eur J Med Chem 1999; 34:729-44; Saito K, et al. Neurosci Lett 1994; 178:211-215; Walsh J L, et al. Brain Res Bull 1994; 33:513-516; and Yates J R, et al. J Neurotrauma 2006, 23:866-881). Published results suggest that 3HAA both inhibits production and reduces toxicity of AO, while QA drives tau hyperphosphorylation leading to NFT formation (FIG. 2). As disclosed herein, it was evaluated whether inhibiting HAAO will reduce production and aggregation of AO by elevating physiological 3HAA, and reduce tau phosphorylation and NFT formation by limiting QA production in the brain using AD disease models.

HAAO is in the right place at the right time. Kynurenine metabolism is segregated by cell type in the brain, with NAD⁺ branch enzymes—including HAAO—expressed by microglia (Alberati-Giani D, et al. J Neurochem 1996, 66:996-1004; and Heyes M P, et al. Biochem J 1996; 320 (Pt 2):595-7) and KA branch enzymes expressed by astrocytes (Guidetti P, et al. Glia 2007, 55:78-92; Guillemin G J, et al. Redox Rep Commun Free Radic Res 2000, 5:108-11; and Guillemin G J, et al. J Neurochem 2001, 78:842-53). One characteristic of AD is elevated neuroinflammation (Heneka M T, et al. Lancet Neurol 2015, 14:388-405; Bronzuoli M R, et al. J Inflamm Res 2016, 9:199-208; and Calsolaro V, Edison P. Alzheimers Dement J Alzheimers Assoc 2016; 12:719-32). Activated microglia localize to amyloid plaques and NFTs, particularly in vulnerable brain regions (Serrano-Pozo A, et al. Am J Pathol 2011, 179:1373-84; Sheffield L G, et al. Neurosci Lett 2000, 285:165-8; Dani M, et al. Brain J Neurol 2018, 141:2740-54; Cras P, et al. Brain Res 1991, 558:312-4; and Bolmont T, et al. J Neurosci 2008, 28:4283-92)

these activated microglia induce indolamine-2,3-dioxygenase (IDO; FIG. 1), driving NAD⁺ branch activity that increases QA production and release into the extracellular environment (Bonda D J, et al. Redox Rep Commun Free Radic Res 2010, 15:161-8; and Schwarcz R, et al. Nat Rev Neurosci 2012, 13:465-77). QA localizes to NFTs in neurons and glial cells near amyloid plaques in brains of AD patients (Guillemin G J, et al. Neuropathol Appl Neurobiol 2005, 31:395-404). High NAD⁺ branch activity in microglia localized to amyloid plaques and NFTs places HAAO in a useful temporal and spatial location to target in AD.

H₂O₂ drives Aβ production from APP. Aβ is produced by the sequential cleavage of APP by β- and γ-secretase (Mattson M P. Nature 2004, 430:631-9). Alternately, α-secretase can cleave APP within the Aβ domain, resulting in non-toxic proteolytic products (FIG. 2) (Mattson M P. Nature 2004, 430:631-9). Brains of AD patients exhibit elevated ROS, particularly near amyloid plaques and NFTs (Ono K, et al. Biochim Biophys Acta 2006, 1762:575-86; and Smith M A. Biochim Biophys Acta 2000, 1502:139-44. H₂O₂ drives Aβ production by promoting the β-secretase proteolytic route (Shen C, et al. J Biol Chem 2008, 283:17721-30) through increased β-secretase (Shen C, et al. J Biol Chem 2008, 283:17721-30; Tamagno E, et al. Neurobiol Dis 2002, 10:279-88; Tamagno E, et al. J Neurochem 2008, 104:683-95; and Jo D-G, et al. Neurobiol Aging 2010, 31:917-25) and APP (Frederikse P H, et al. J Biol Chem 1996; 271:10169-74). expression. Reducing H₂O₂ in the brain can also serve as a target for treating or preventing one or more symptoms of AD or AD-related pathology. As disclosed herein, it was found that 3HAA directly degrades H₂O₂ in vitro in C. elegans, providing a potential mechanism for reducing Aβ toxicity. Whether 3HAA can affect H₂O₂ levels or APP processing in cell culture and in mice will be investigated.

3HAA directly binds Aβ and prevents aggregation in vitro. Aβ misfolding occurs as a conversion from a nontoxic α-helical or random coil conformation to a neurotoxic and aggregate-prone β-helical conformation (Yoda M, et al. Biochem Biophys Res Commun 2008; 376:56-9). The HHQK region of Aβ is important to this conformational change (Giulian D, et al. J Biol Chem 1998, 273:29719-26; and Meek et al. J Psychiatry Neurosci 2013; 38:269-75) identified 3HAA in a computational screen for small molecules capable of directly binding the HHQK domain, and subsequently demonstrated that 3HAA prevents Aβ aggregation in buffered media. Whether 3HAA can inhibit Aβ aggregation in both cultured cells and in mice will be evaluated.

QA drives tau phosphorylation. Human neurons do not produce QA endogenously, but import it from the extracellular environment (Guillemin G J, et al. J Neurosci Off Soc Neurosci 2007, 27:12884-92; and Guillemin G J, et al. Glia 2005, 49:15-23) QA localizes to NFTs in the brains of AD patients and drives tau phosphorylation in cultured human neurons (Rahman A, et al. PLoS ONE 2009, 4:e6344). This mechanism will be tested in vivo.

QA seeds α-synuclein aggregation. QA self-organizes into fibrils that seed and accelerate aggregation of α-synuclein, which forms toxic aggregates in Parkinson's Disease (Tavassoly O, et al. J Mol Biol 2018). Thus, whether QA may similarly drive aggregation of Aβ and tau in AD will be tested. As disclosed herein, the enzyme 3-hydroxyanthranilic acid dioxygenase (HAAO) will be evaluated as a target to simultaneously improve two hallmark AD pathologies: (1) production and aggregation of beta-amyloid (Aβ) into extracellular plaques and (2) hyperphosphorylation and aggregation of tau into neurofibrillary tangles (NFTs). HAAO catalyzes the conversion of the metabolite 3-hyrdoxyanthranilic acid (3HAA) to quinolinic acid (QA) in the tryptophan-kynurenine metabolic pathway. Inhibition of HAAO results in elevated physiological 3HAA and reduced QA. Described herein are methods of both increasing 3HAA and decreasing QA to treat and/or prevent or improve AD pathology.

The Examples provide results showing that supplementation with 3HAA or inhibition of HAAO robustly extended lifespan and delayed Aβ pathology in Caenorhabditis elegans. These benefits were mediated, at least in part, by direct degradation of hydrogen peroxide (H₂O₂) by 3HAA. Reactive oxygen species (ROS) are elevated both with age and in the brains of AD patients and H₂O₂ drives production of Aβ via proteolytic processing of amyloid precursor protein (APP) (Shen C, et al. J Biol Chem 2008, 283:17721-30; Tamagno E, et al. Neurobiol Dis 2002, 10:279-88; Tamagno E, et al. J Neurochem 2008, 104:683-95; Jo D-G, et al. Neurobiol Aging 2010, 31:917-25; and Frederikse P H, et al. J Biol Chem 1996, 271:10169-74). 3HAA also directly binds Aβ, preventing misfolding and aggregation in vitro (Meek A, et al. J Psychiatry Neurosci 2013, 38:269-75). Elevating 3HAA can reduce both production and toxicity of Aβ. In contrast to 3HAA, QA drives tau phosphorylation in cultured human neurons, an early step in the formation of NFTs (Rahman A, et al. PLoS ONE 2009, 4:e6344). QA also self-assembles into fibrillary structures capable of seeding aggregation of α-synuclein, the major constituent of Lewy bodies in Parkinson's disease (Tavassoly O, et al. J Mol Biol 2018). Thus, QA assemblies may seed aggregation of other proteins, including Aβ and tau. As also disclosed herein, methods of reducing QA can serve to both remove a driver of tau phosphorylation and a seed for aggregate formation during AD. These observations have been made using 3HAA or QA either in buffered media, in C. elegans, or in cell culture. As described in the Examples, it will be tested whether inhibiting HAAO will mitigate amyloid and tau toxicity via elevated 3HAA and reduced QA. Also, in vitro molecular tools will be combined with cellular and mouse models of amyloid and tau pathology to test and examine the mediating mechanisms linking 3HAA to amyloid toxicity and QA to tau toxicity.

Stress Resistance

Organisms are subjected to constant and varied stress and respond by activating a range of cellular stress response pathways. The organization of individual stress response pathways and response to specific stressors has been extensively studied. In contrast, the molecular response to multiple simultaneous forms of stress, and the mechanisms of interaction and feedback between distinct stress response pathways, are less well understood. The fundamental principles of multiple stress response are broadly relevant to human health and disease. Many forms of stress increase with age, and different disease states are characterized by distinct combinations of cellular stress and activation/repression of different response pathways. As described herein the combination of tools in systems and comparative genetics can be used to systematically (1) characterize the physiological and molecular response to different combinations of stress, (2) build a comprehensive gene interaction network for selected stress response pathways, and (3) investigate the detailed molecular mechanisms behind genes and pathways involved in response to multiple forms of stress.

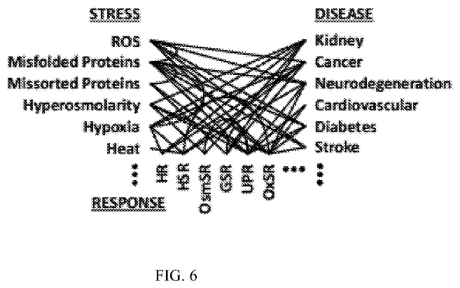

Organisms experience a range of stresses arising from their environment or from internal metabolic processes. How cells respond to varied and changing combinations of stressors is important to long-term health and survival. Eukaryotic cells have evolved a variety of molecular systems for dealing with the deleterious effects of different stresses (FIG. 6). How organisms respond to stress is a fundamental question in biology, and is relevant to human health. Most forms of cellular stress are elevated with increasing age and human diseases are characterized by elevated stress or impaired ability to cope with specific forms of stress (FIG. 6). While substantial work has been done to understand how cells respond to specific stressors, a knowledge gap exists around how stress response systems respond to when challenged with multiple forms of stress in combination.

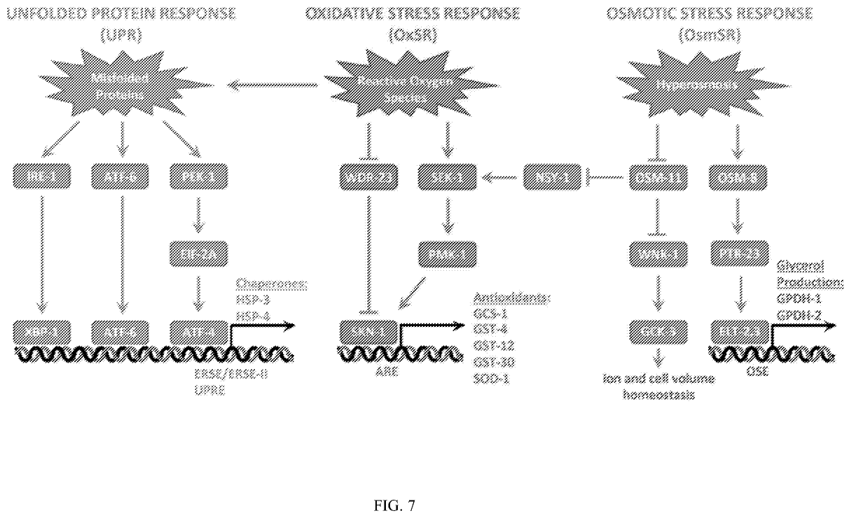

How animals respond when challenged with multiple forms of stress is less well understood. There is a particular need to examine stress combinations that commonly arise in human disease. In contrast, the field of toxicology has produced a wealth of data on the interaction between “natural” stressors (e.g. temperature, desiccation) and environmental chemicals (e.g. heavy metals, pesticides), primarily in insects and aquatic species. Striking synergistic toxicity is often observed when multiple forms of stress are present. In agriculture, crops are often exposed to combinations of environmental stress (e.g. heat and drought; cold and intense light) that can dramatically affect crop yield. The majority of transcriptional changes observed in Arabidopsis thaliana exposed to combined stress are not predicted by the response to the individual stresses. Based on these studies, understanding the response of an organism to a single stress is likely insufficient to predict the response to multiple stressors. In mammals, a body of work examines the impact of heat acclimation on various forms of physiological stress, while other stress combinations are largely unexplored. The nematode Caenorhabditis elegans responds to different forms of stress by activating many of the same evolutionarily-conserved molecular pathways as mammals (FIG. 7). Because they are also easy and inexpensive to culture, they have been used extensively to model response to individual stressors. As with mammals, a few examples of combined stress have been published. To address this deficit, C. elegans will be used to profile the response to range of combined stressors.

Organisms exposed to mild stress often become resistant to a subsequent toxic exposure to the same stress (hormesis) or a different stress (cross-adaptation). This resistance is conferred by persistent activation of stress response pathways by the initial mild stress. C. elegans display hormetic adaptation to a wide range of stresses—oxidative, heat, heavy metals, endoplasmic reticulum (ER)—and cross-adaptation in the few cases examined. For example, pre-exposure to hyperoxia confers protection against X-irradiation and the pro-oxidant agent juglone, while mild UV exposure confers protection against metal toxicity. Cross-adaptation is not always beneficial; pre-exposure to mild-heat stress confers resistance to mercury toxicity, but sensitizes worms to pro-oxidant acrylamide. Combining multiple forms of hormesis is an area that remains completely unexplored. It will be tested whether combining different mild stressors with distinct hormetic responses will confer broad spectrum stress resistance. To test this, cross-adaptation will be systematically evaluated across a range of stressors in C. elegans.

Cells respond to different forms of stress by activating a range of stress response pathways, forming a molecular network of interconnected genetic modules (FIG. 7). Some modules are well defined—the unfolded protein response (UPR), heat shock response (HSR), oxidative stress response (OxSR), and hypoxic response (HR)—while others, such as the osmotic stress response (OsmSR) and Golgi stress response (GSR) are being investigated. Different stressors produce distinct patterns of activation or repression in various response pathways, and the regulation of response pathways is not independent (e.g. the OxSR is inhibited by the HSR5). Described herein are experiments to define the structure and dynamics of this network, identify new genetic players in the stress response, and determine how modules interact in response to different types and combinations of stress.

Human diseases are characterized by upregulation of different combinations of stress and/or dysregulation of different stress response pathways (FIG. 6). For example, Alzheimer's disease is characterized by elevated oxidative stress, increased inflammation, and an increased burden of misfolded and aggregated proteins, while cancer is characterized by increased burden of DNA damage, impaired DNA repair, elevated oxidative stress, and localized hypoxia. By defining the structure of the stress response network, important nodes that can be targeted to increase resistance to specific combinations of stress can be identified. Targeted mechanistic studies on the genes and small molecules that form these nodes will be conducted with the goal of understanding how distinct combinations of stress manifest in different disease states, and how these nodes can be manipulated to improve resistance to different combinations of stress.

As disclosed herein, the results from the mechanistic studies reveal that inhibiting the KP enzyme HAAO increases systemic resistance to both oxidative and misfolded protein stress. As organisms age, many forms of cellular stress increase while the efficacy of stress response pathways declines. Genes with age-dependent expression are thus good candidates for causal factors in multi-stress response. In conducting this work, it was discovered that there are a surprising lack of studies examining combined stress in animal models of human disease. To this end, this disclosure describes the following: (1) Identify candidate stress-response gene sets from genome-scale human data and use direct RNAi screening and genetic network building in C. elegans to select priority candidates; (2) Molecular characterization, characterize mechanisms of altered stress response for priority candidates in C. elegans; and (3) Validation, validate mechanistic models in mice.

This approach leverages the strengths of three model systems to efficiently identify and characterize genes involved in multi-stress response by beginning with gene sets already of interest to human health (Phase 1), employing high-throughput, low-cost genetic tools in C. elegans to rapidly identify and characterize the most interesting candidate genes (Phase 2), and validating the most interesting candidates in mice (Phase 3).

Methods of Treatment

Disclosed herein are methods of preventing or reducing oxidative stress in a subject. The method comprising administering to the subject a therapeutically effective amount of a composition that: a) reduces expression of HAAO; b) reduces expression of HAAO; c) inhibits HAAO activity; d) increases the bioavailability 3HAA; d) directly supplements 3HAA; f) degrades QA; g) increases expression of quinolinate phosphoribosyltransferase (QPRT); h) increases expression of QPRT; i) activates QPRT; or j) two or more of a) to i) in combination, thereby preventing or reducing oxidative stress in the subject.

Disclosed herein are methods of preventing one or more proteins from misfolding or aggregating in a subject. The method comprising administering to the subject a therapeutically effective amount of a composition that: a) reduces expression of HAAO; b) reduces expression of HAAO; c) inhibits HAAO activity; d) increases the bioavailability 3HAA; d) directly supplements 3HAA; f) degrades QA; g) increases expression of QPRT; h) increases expression of QPRT; i) activates QPRT; or j) two or more of a) to i) in combination, thereby preventing one or more proteins from misfolding or protein aggregation.

Disclosed herein are methods of promoting proper folding of one or more proteins. The method comprising administering a composition that: a) reduces expression of HAAO; b) reduces expression of HAAO; c) inhibits HAAO activity; d) increases the bioavailability 3HAA; d) directly supplements 3HAA; f) degrades QA; g) increases expression of QPRT; h) increases expression of QPRT; i) activates QPRT; or j) two or more of a) to i) in combination, thereby promoting proper folding of one or more proteins.

Disclosed herein are methods of increasing H₂O₂ breakdown. The method comprising administering a composition that: a) reduces expression of HAAO; b) reduces expression of HAAO; c) inhibits HAAO activity; d) increases the bioavailability 3HAA; d) directly supplements 3HAA; f) degrades QA; g) increases expression of QPRT; h) increases expression of QPRT; i) activates QPRT; or j) two or more of a) to i) in combination, thereby increasing H₂O₂ breakdown.

Disclosed herein are methods of increasing or extending a subject's lifespan. The method comprising administering a composition that: a) reduces expression of HAAO; b) reduces expression of HAAO; c) inhibits HAAO activity; d) increases the bioavailability 3HAA; d) directly supplements 3HAA; f) degrades QA; g) increases expression of QPRT; h) increases expression of QPRT; i) activates QPRT; or j) two or more of a) to i) in combination, thereby increasing or extending the subject's lifespan.

Disclosed herein are methods of delaying or preventing the decline in a subject's physical health with age. The method comprising administering a composition that: a) reduces expression of HAAO; b) reduces expression of HAAO; c) inhibits HAAO activity; d) increases the bioavailability 3HAA; d) directly supplements 3HAA; f) degrades QA; g) increases expression of QPRT; h) increases expression of QPRT; i) activates QPRT; or j) two or more of a) to i) in combination, thereby delaying or preventing the decline in the subject's physical health with age.

Disclosed herein are methods of increasing 3HAA in a subject. The method comprising administering a composition that: a) reduces expression of HAAO; b) reduces expression of HAAO; c) inhibits HAAO activity; d) increases the bioavailability 3HAA; d) directly supplements 3HAA; f) degrades QA; g) increases expression of QPRT; h) increases expression of QPRT; i) activates QPRT; or j) two or more of a) to i) in combination, thereby increasing 3HAA in a subject.

Disclosed herein are methods of decreasing QA in a subject. The method comprising administering a composition that: a) reduces expression of HAAO; b) reduces expression of HAAO; c) inhibits HAAO activity; d) increases the bioavailability 3HAA; d) directly supplements 3HAA; f) degrades QA; g) increases expression of QPRT; h) increases expression of QPRT; i) activates QPRT; or j) two or more of a) to i) in combination, thereby decreasing QA in a subject.

Disclosed herein are methods of reducing amyloid-beta production, misfolding, or aggregation in a subject. The method comprising administering to the subject a composition that: a) reduces expression of HAAO; b) reduces expression of HAAO; c) inhibits HAAO activity; d) increases the bioavailability 3HAA; d) directly supplements 3HAA; f) degrades QA; g) increases expression of QPRT; h) increases expression of QPRT; i) activates QPRT; or j) two or more of a) to i) in combination, thereby reducing amyloid-beta production, misfolding, or aggregation in a subject.

Disclosed herein are methods of preventing or reducing neurofibrillary tangles in a subject. The method comprising administering to the subject a composition that: a) reduces expression of HAAO; b) reduces expression of HAAO; c) inhibits HAAO activity; d) increases the bioavailability 3HAA; d) directly supplements 3HAA; f) degrades QA; g) increases expression of QPRT; h) increases expression of QPRT; i) activates QPRT; or j) two or more of a) to i) in combination, thereby preventing or reducing neurofibrillary tangles in the subject.

Disclosed herein are methods of preventing protein misfolding or protein aggregation in a subject. The method comprising administering to the subject in need thereof a therapeutically effective amount of a composition that: a) reduces expression of HAAO; b) reduces expression of HAAO; c) inhibits HAAO activity; d) increases the bioavailability 3HAA; d) directly supplements 3HAA; f) degrades QA; g) increases expression of QPRT; h) increases expression of QPRT; i) activates QPRT; or j) two or more of a) to i) in combination, thereby preventing protein misfolding or protein aggregation in the subject.

Disclosed herein are methods of delaying or preventing cognitive decline in a subject with Alzheimer's disease. The method comprising administering to the subject in need thereof a therapeutically effective amount of a composition that: a) reduces expression of HAAO; b) reduces expression of HAAO; c) inhibits HAAO activity; d) increases the bioavailability 3HAA; d) directly supplements 3HAA; f) degrades QA; g) increases expression of QPRT; h) increases expression of QPRT; i) activates QPRT; or j) two or more of a) to i) in combination, thereby delaying or preventing cognitive decline in a subject with Alzheimer's disease.

Disclosed herein are methods of delaying or preventing cognitive decline in a subject with Huntington's disease. The method comprising administering to the subject in need thereof a therapeutically effective amount of a composition that: a) reduces expression of HAAO; b) reduces expression of HAAO; c) inhibits HAAO activity; d) increases the bioavailability 3HAA; d) directly supplements 3HAA; f) degrades QA; g) increases expression of QPRT; h) increases expression of QPRT; i) activates QPRT; or j) two or more of a) to i) in combination, thereby delaying or preventing cognitive decline in a subject with Huntington's disease.

Disclosed herein are methods of reducing inflammation in a subject. The method comprising administering to the subject in need thereof a therapeutically effective amount of a composition that: a) reduces expression of HAAO; b) reduces expression of HAAO; c) inhibits HAAO activity; d) increases the bioavailability 3HAA; d) directly supplements 3HAA; f) degrades QA; g) increases expression of QPRT; h) increases expression of QPRT; i) activates QPRT; or j) two or more of a) to i) in combination, thereby reducing inflammation in the subject.

Disclosed herein are methods of increasing 3-hydroxyanthranilic acid (3HAA) in a subject. In some aspects, the method comprising administering to the subject a composition that: a) reduces expression of 3-hydroxyanthranilic acid dioxygenase (HAAO); b) reduces expression of HAAO; c) inhibits HAAO activity d) increases the bioavailability 3-hydroxyanthranilic acid (3HAA); e) directly supplements 3HAA; or f) two or more of a) to e) in combination, thereby increasing 3HAA in the subject. In some aspects, the composition that reduces expression of HAAO, reduces expression of HAAO, or inhibits HAAO activity can be a 3HAA analog, 4CL-3AA or 6CL-TRP. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the subject can be a human. In some aspects, the subject can be diagnosed with, can be at risk for, or has Alzheimer's disease. In some aspects, the subject can be diagnosed with, can be at risk for, or has multiple age-associated diseases, e.g. cardiovascular disease, diabetes, neurodegenerative disease, chronic kidney disease (aka co-morbidities). In some aspects, the subject can be diagnosed with, can be at risk for, or has Huntington's disease.

Disclosed herein are methods of decreasing QA in a subject. In some aspects, the method comprises administering to the subject a composition that: a) reduces expression of 3-hydroxyanthranilic acid dioxygenase (HAAO); b) reduces expression of HAAO; or c) inhibits HAAO activity; d) degrades QA; e) increases expression of QPRT; f) increases expression of QPRT; g) activates QPRT; h) two or more of a) to g) in combination, thereby decreasing QA in the subject. In some aspects, the composition that reduces expression of HAAO, reduces expression of HAAO, or inhibits HAAO activity can be a 3HAA analog, 4CL-3AA or 6CL-TRP. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the composition that increases expression of QPRT, increases expression of QPRT, or activates QPRT activity can be selected from the group consisting of a small molecule, a recombinant protein, an mRNA, and combinations thereof. In some aspects, the subject can be a human. In some aspects, the subject can be diagnosed with, can be at risk for, or has Alzheimer's disease. In some aspects, the subject can be diagnosed with, can be at risk for, or has multiple age-associated diseases, e.g. cardiovascular disease, diabetes, neurodegenerative disease, chronic kidney disease (aka co-morbidities). In some aspects, the subject can be diagnosed with, can be at risk for, or has Huntington's disease.

Disclosed herein are methods of preventing or reducing oxidative stress in a subject. In some aspects, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby preventing or reducing oxidative stress in the subject.

Disclosed herein are methods of preventing one or more proteins from misfolding or aggregating in a subject. In some aspects, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby preventing one or more proteins from misfolding or aggregating in a subject. In some aspects, the administration of the composition can increase or lead to increase break down (or decomposition) of hydrogen peroxide. In some aspects, the composition can be administered to a subject in need thereof. In some aspects, the composition that reduces expression of HAAO, reduces expression of HAAO, or inhibits HAAO activity can be a 3HAA or tryptophan analog, e.g. 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the composition that increases expression of QPRT, increases expression of QPRT, or activates QPRT activity can be selected from the group consisting of a small molecule, a recombinant protein, an mRNA, and combinations thereof. In some aspects, the subject can be a human. In some aspects, the subject can be diagnosed with, can be at risk for, or has Alzheimer's disease. In some aspects, the subject can be diagnosed with, can be at risk for, or has multiple age-associated diseases, e.g. cardiovascular disease, diabetes, neurodegenerative disease, chronic kidney disease (aka co-morbidities).

Disclosed herein are methods of promoting proper folding of one or more proteins in a subject. In some aspects, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby promoting proper folding of one or more proteins in a subject. In some aspects, the administration of the composition can increase or lead to increase break down (or decomposition) of hydrogen peroxide. In some aspects, the composition can be administered to a subject in need thereof. In some aspects, the composition that reduces expression of HAAO, reduces expression of HAAO, or inhibits HAAO activity can be a 3HAA or tryptophan analog, e.g. 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the composition that increases expression of QPRT, increases expression of QPRT, or activates QPRT activity can be selected from the group consisting of a small molecule, a recombinant protein, an mRNA, and combinations thereof. In some aspects, the subject can be a human. In some aspects, the subject can be diagnosed with, can be at risk for, or has Alzheimer's disease. In some aspects, the subject can be diagnosed with, can be at risk for, or has multiple age-associated diseases, e.g. cardiovascular disease, diabetes, neurodegenerative disease, chronic kidney disease (aka co-morbidities).

Disclosed herein are methods of increasing hydrogen peroxide (H₂O₂) breakdown in a subject. In some aspects, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby increasing H₂O₂ breakdown in a subject. In some aspects, the administration of the composition can increase or lead to increase break down (or decomposition) of hydrogen peroxide. In some aspects, the composition can be administered to a subject in need thereof. In some aspects, the composition that reduces expression of HAAO, reduces expression of HAAO, or inhibits HAAO activity can be a 3HAA or tryptophan analog, e.g. 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the composition that increases expression of QPRT, increases expression of QPRT, or activates QPRT activity can be selected from the group consisting of a small molecule, a recombinant protein, an mRNA, and combinations thereof. In some aspects, the subject can be a human. In some aspects, the subject can be diagnosed with, can be at risk for, or has Alzheimer's disease. In some aspects, the subject can be diagnosed with, can be at risk for, or has multiple age-associated diseases, e.g. cardiovascular disease, diabetes, neurodegenerative disease, chronic kidney disease (aka co-morbidities).

Disclosed herein are methods of delaying or preventing the decline in a subject's physical health with age. In some aspects, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby delaying or preventing the decline in a subject's physical health with age. In some aspects, the composition can be administered to a subject in need thereof. In some aspects, the composition that reduces expression of HAAO, reduces expression of HAAO, or inhibits HAAO activity can be a 3HAA or tryptophan analog, e.g. 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the composition that increases expression of QPRT, increases expression of QPRT, or activates QPRT activity can be selected from the group consisting of a small molecule, a recombinant protein, an mRNA, and combinations thereof. In some aspects, the subject can be a human. In some aspects, the subject can be diagnosed with, can be at risk for, or has Alzheimer's disease. In some aspects, the subject can be diagnosed with, can be at risk for, or has multiple age-associated diseases, e.g. cardiovascular disease, diabetes, neurodegenerative disease, chronic kidney disease (aka co-morbidities).

Disclosed herein are methods of reducing amyloid-beta production, misfolding, or aggregation in a subject. In some aspects, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby reducing amyloid-beta production, misfolding, or aggregation in a subject. In some aspects, the composition can be administered to a subject in need thereof. In some aspects, the composition that reduces expression of HAAO, reduces expression of HAAO, or inhibits HAAO activity can be a 3HAA or tryptophan analog, e.g. 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the composition that increases expression of QPRT, increases expression of QPRT, or activates QPRT activity can be selected from the group consisting of a small molecule, a recombinant protein, an mRNA, and combinations thereof. In some aspects, the subject can be a human. In some aspects, the subject can be diagnosed with, can be at risk for, or has Alzheimer's disease. In some aspects, the subject can be diagnosed with, can be at risk for, or has multiple age-associated diseases, e.g. cardiovascular disease, diabetes, neurodegenerative disease, chronic kidney disease (aka co-morbidities).

Disclosed herein are methods of delaying or preventing cognitive decline in a subject with Alzheimer's disease. In some aspects, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby delaying or preventing cognitive decline in a subject with Alzheimer's disease. In some aspects, the composition can be administered to a subject in need thereof. In some aspects, the composition that reduces expression of HAAO, reduces expression of HAAO, or inhibits HAAO activity can be a 3HAA or tryptophan analog, e.g. 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the composition that increases expression of QPRT, increases expression of QPRT, or activates QPRT activity can be selected from the group consisting of a small molecule, a recombinant protein, an mRNA, and combinations thereof. In some aspects, the subject can be a human. In some aspects, the subject can be diagnosed with, can be at risk for, or has Alzheimer's disease. In some aspects, the subject can be diagnosed with, can be at risk for, or has multiple age-associated diseases, e.g. cardiovascular disease, diabetes, neurodegenerative disease, chronic kidney disease (aka co-morbidities).

Disclosed herein are methods of delaying or preventing cognitive decline in a subject with Huntington's disease. In some aspects, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby delaying or preventing cognitive decline in a subject with Huntington's disease. In some aspects, the composition can be administered to a subject in need thereof. In some aspects, the composition that reduces expression of HAAO, reduces expression of HAAO, or inhibits HAAO activity can be a 3HAA or tryptophan analog, e.g. 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the composition that increases expression of QPRT, increases expression of QPRT, or activates QPRT activity can be selected from the group consisting of a small molecule, a recombinant protein, an mRNA, and combinations thereof. In some aspects, the subject can be a human. In some aspects, the subject can be diagnosed with, can be at risk for, or has Huntington's disease. In some aspects, the subject can be diagnosed with, can be at risk for, or has Alzheimer's disease. In some aspects, the subject can be diagnosed with, can be at risk for, or has multiple age-associated diseases, e.g. cardiovascular disease, diabetes, neurodegenerative disease, chronic kidney disease, Alzheimer's disease (aka co-morbidities).

Disclosed herein are methods of preventing or reducing neurofibrillary tangles in a subject. In some aspects, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby preventing or reducing neurofibrillary tangles in a subject. In some aspects, the composition can be administered to a subject in need thereof. In some aspects, the composition that reduces expression of HAAO, reduces expression of HAAO, or inhibits HAAO activity can be a 3HAA or tryptophan analog, e.g. 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the composition that increases expression of QPRT, increases expression of QPRT, or activates QPRT activity can be selected from the group consisting of a small molecule, a recombinant protein, an mRNA, and combinations thereof. In some aspects, the subject can be a human. In some aspects, the subject can be diagnosed with, can be at risk for, or has Alzheimer's disease. In some aspects, the subject can be diagnosed with, can be at risk for, or has multiple age-associated diseases, e.g. cardiovascular disease, diabetes, neurodegenerative disease, chronic kidney disease (aka co-morbidities).

Disclosed herein are methods of treating a disease or a condition associated with misfolding of a protein or protein aggregation in a subject. In some aspects, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby treating a disease or a condition associated with misfolding of a protein or protein aggregation in a subject. In some aspects, the composition can be administered to a subject in need thereof. In some aspects, the composition that reduces expression of HAAO, reduces expression of HAAO, or inhibits HAAO activity can be a 3HAA or tryptophan analog, e.g. 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the composition that increases expression of QPRT, increases expression of QPRT, or activates QPRT activity can be selected from the group consisting of a small molecule, a recombinant protein, an mRNA, and combinations thereof. In some aspects, the subject can be a human. In some aspects, the subject can be diagnosed with, can be at risk for, or has Alzheimer's disease. In some aspects, the subject can be diagnosed with, can be at risk for, or has multiple age-associated diseases, e.g. cardiovascular disease, diabetes, neurodegenerative disease, chronic kidney disease (aka co-morbidities).

Disclosed herein are methods of reducing inflammation in a subject. In some aspects, the method comprising administering to the subject a therapeutically effective amount of a composition that: a) reduces expression of HAAO; b) reduces expression of HAAO; c) inhibits HAAO activity d) increases the bioavailability 3HAA; e) directly supplements 3HAA; f) degrades QA; g) increases expression of QPRT; h) increases expression of QPRT; i) activates QPRT; or j) two or more of a) to i) in combination, thereby reducing inflammation in a subject. In some aspects, the composition can be administered to a subject in need thereof. In some aspects, the composition that reduces expression of HAAO, reduces expression of HAAO, or inhibits HAAO activity can be a 3HAA or tryptophan analog, e.g. 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the composition that increases expression of QPRT, increases expression of QPRT, or activates QPRT activity can be selected from the group consisting of a small molecule, a recombinant protein, an mRNA, and combinations thereof. In some aspects, the subject can be a human. In some aspects, the subject can be diagnosed with, can be at risk for, or has Alzheimer's disease. In some aspects, the subject can be diagnosed with, can be at risk for, or has multiple age-associated diseases, e.g. cardiovascular disease, diabetes, neurodegenerative disease, chronic kidney disease (aka co-morbidities).

Further disclosed herein, are methods directed to reducing the expression, level or activity of one or more genes or proteins, wherein the one or more genes or proteins include but are not limited to kynu-1, tdo-2, and haao-1. Also disclosed herein are compositions that can be administered to a subject to reduce the expression, level or activity of one or more genes or proteins, wherein the one or more genes or proteins include but are not limited to kynu-1, tdo-2, and haao-1.

In some aspects, the composition that increases expression of QPRT, increases expression of QPRT, or activates QPRT activity can be selected from the group consisting of a small molecule, a recombinant protein, an mRNA, and combinations thereof.

In some aspects, in any of the methods disclosed herein, the subject can be a human. In some aspects, the subject can be diagnosed with, can be at risk for, or has Alzheimer's disease. In some aspects, the subject can be diagnosed with, can be at risk for, or has one or more multiple age-associated diseases, e.g. cardiovascular disease, diabetes, neurodegenerative disease, chronic kidney disease (also known as co-morbidities). In some aspects, the subject can be diagnosed with, can be at risk for, or has Huntington's disease.

Disclosed herein are methods of preventing oxidative stress in a subject. Also disclosed herein are method of reducing oxidative stress in a subject. In some aspects, the method comprises administering to the subject a therapeutically effective amount of a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby preventing (or reducing) oxidative stress in the subject. In some aspects, the administration of the composition can increase or lead to increase breakdown (or decomposition) of hydrogen peroxide. In some aspects, the composition can be administered to a subject in need thereof. In some aspects, the composition that reduces expression of HAAO, reduces expression of HAAO, or inhibits HAAO activity can be a 3HAA or tryptophan analog, e.g. 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the subject can be a human. In some aspects, the subject can be diagnosed with, can be at risk for, or has Alzheimer's disease. In some aspects, the subject can be diagnosed with, can be at risk for, or has one or more multiple age-associated diseases, e.g., cardiovascular disease, diabetes, neurodegenerative disease, chronic kidney disease (also known as co-morbidities). Disclosed herein are methods of preventing one or more proteins from misfolding or aggregating. In some aspects, the method comprises administering a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby preventing one or more proteins from misfolding or protein aggregation.

In some aspects, the composition can be administered to a subject in need thereof. In some aspects, the subject can be a human. In some aspects, the subject can be diagnosed with, can be at risk for, or has Alzheimer's disease. In some aspects, the subject can be diagnosed with, can be at risk for, or has Huntington's disease. In some aspects, the composition that reduces expression of HAAO, reduces expression of HAAO, or inhibits HAAO activity can be a 3HAA analog, 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof.

Disclosed herein are methods of promoting proper folding of one or more proteins. In some aspects, the method comprises administering a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby promoting proper folding of one or more proteins. In some aspects, the composition can be administered to a subject in need thereof. In some aspects, the subject can be a human. In some aspects, the subject can be diagnosed with, can be at risk for, or has Alzheimer's disease. In some aspects, the composition that reduces expression of HAAO, reduces expression of HAAO, or inhibits HAAO activity can be a 3HAA or tryptophan analog, 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof.

Disclosed herein are methods of increasing H₂O₂ breakdown. In some aspects, the method comprises administering a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby increasing H₂O₂ degradation. In some aspects, the composition can be administered to a subject in need thereof. In some aspects, the subject can be a human. In some aspects, the subject can be diagnosed with, can be at risk for, or has Alzheimer's disease. In some aspects, the composition that reduces expression of HAAO, reduces expression of HAAO, or inhibits HAAO activity can be a 3HAA or tryptophan analog, 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof.

Disclosed herein are methods of increasing or extending a subject's lifespan. In some aspects, the method comprises administering a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby increasing or extending the subject's lifespan. In some aspects, the composition can be administered to a subject in need thereof. In some aspects, the subject can be a worm. In some aspects, the subject can be a human. In some aspects, the subject can be diagnosed with, can be at risk for, or has Alzheimer's disease. In some aspects, the subject can be diagnosed with, can be at risk for, or has Huntington's disease. In some aspects, the composition that reduces expression of HAAO, reduces expression of HAAO, or inhibits HAAO activity can be a 3HAA or tryptophan analog, 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof.

Disclosed herein are methods of delaying paralysis in a subject expressing amyloid-beta in body wall muscle. In some aspects, the method comprises administering to the subject a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby delaying paralysis in the subject. In some aspects, the subject can be a worm. In some aspects, the subject can be a human. In some aspects, the subject can be diagnosed with, can be at risk for, or has Alzheimer's disease. In some aspects, the composition that reduces expression of HAAO, reduces expression of HAAO, or inhibits HAAO activity can be a 3HAA analog, 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof.

Disclosed herein are methods of delaying paralysis in a subject. In some aspects, the subject expresses or is identified as expressing a 35-unit polyglutamine repeat peptide in body wall muscle. In some aspects, the method comprises administering to the subject a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby delaying paralysis in the subject. In some aspects, the subject can be a worm. In some aspects, the subject can be a human. In some aspects, the subject can be diagnosed with, can be at risk for, or has Huntington's disease. In some aspects, the composition that reduces expression of HAAO, reduces expression of HAAO, or inhibits HAAO activity can be a 3HAA analog, 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof.

Disclosed herein are methods of decreasing quinolinic acid in a subject. In some aspects, the method comprises administering to the subject a composition that: a) reduces expression of 3-hydroxyanthranilic acid dioxygenase (HAAO); b) reduces expression of HAAO; or c) inhibits HAAO activity; thereby decreasing quinolinic acid in the subject. In some aspects, the composition that reduces expression of HAAO, reduces expression of HAAO, or inhibits HAAO activity can be a 3HAA analog, 4CL-3AA or 6CL-TRP. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the subject can be a human. In some aspects, the subject can be diagnosed with, can be at risk for, or has Alzheimer's disease.

Disclosed herein are methods of preventing or reducing neurofibrillary tangles in a subject. In some aspects, the method comprises administering to the subject a composition that: a) reduces expression of 3-hydroxyanthranilic acid dioxygenase (HAAO); b) reduces expression of HAAO; c) inhibits HAAO activity; d) decrease the concentration of quinolinic acid (QA); e) increases the bioavailability 3-hydroxyanthranilic acid (3HAA); f) increases the concentration of 3-hydroxyanthranilic acid (3HAA); or g) one or more of a) to c) in combination with d), e) and/or f), thereby preventing or reducing neurofibrillary tangles in the subject. In some aspects, the composition that reduces expression of HAAO, reduces expression of HAAO, or inhibits HAAO activity can be a 3HAA analog, 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the subject can be a human. In some aspects, the subject can be diagnosed with, can be at risk for, or has Alzheimer's disease.

Disclosed herein are methods of preventing protein misfolding or protein aggregation in a subject. In some aspects, the method comprises administering to the subject in need thereof a therapeutically effective amount of a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby preventing protein misfolding or protein aggregation in the subject. In some aspects, the composition that reduces expression of HAAO, reduces expression of HAAO, or inhibits HAAO activity can be a 3HAA analog, 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the subject can be a human. In some aspects, the subject can be diagnosed with, can be at risk for, or has Alzheimer's disease.

Disclosed herein are methods of treating a disease or a condition associated with misfolding of a protein or protein aggregation in a subject. In some aspects, the method comprises administering to the subject in need thereof a therapeutically effective amount of a composition that: a) reduces expression of 3-hydroxyanthranilic acid dioxygenase (HAAO); b) reduces expression of MAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby treating a disease or a condition associated with misfolding of a protein or protein aggregation in the subject. In some aspects, the disease or condition is Alzheimer's disease. In some aspects, the disease or condition is Huntington's disease. In some aspects, the composition that reduces expression of HAAO, reduces expression of HAAO, or inhibits HAAO activity can be a 3HAA analog, 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the subject can be a human. In some aspects, the subject can be diagnosed with, can be at risk for, or has Alzheimer's disease.

Disclosed herein are methods of reducing amyloid-beta production in a subject. In some aspects, the method comprises administering to the subject in need thereof a therapeutically effective amount of a composition that degrades H₂O₂ or increases degradation of H₂O₂. In some aspects, the composition further: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) increases the bioavailability and concentration of 3-hydroxyanthranilic acid (3HAA). In some aspects, the composition that reduces expression of HAAO, reduces expression of HAAO, or inhibits HAAO activity can be a 3HAA analog, 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the subject can be a human. In some aspects, the subject can be diagnosed with, can be at risk for, or has Alzheimer's disease. In some aspects, the composition further g) increases expression of QPRT; h) increases expression of QPRT; i) activates QPRT; or j) degrades QA; or two or more of a) to j) in combination.

Disclosed herein are methods of preventing oxidative stress or increasing H₂O₂ degradation in a subject. In some aspects, the method comprises administering to the subject in need thereof a therapeutically effective amount of a composition that: a) reduces expression of 3-hydroxyanthranilic acid dioxygenase (HAAO); b) reduces expression of HAAO; c) inhibits HAAO activity; d) increases the bioavailability 3-hydroxyanthranilic acid (3HAA); e) increases the concentration of 3-hydroxyanthranilic acid (3HAA); or f) one or more of a) to c) in combination with d) and/or e), thereby preventing oxidative stress or increasing H₂O₂ degradation in the subject. In some aspects, the composition that reduces expression of HAAO, reduces expression of HAAO, or inhibits HAAO activity can be a 3HAA analog, 4CL-3AA or 6CL-TRP. In some aspects, the composition that increases or enhances the bioavailability or concentration of 3HAA can be a 3HAA supplement or a 3HAA variant or analog thereof. In some aspects, the composition that reduces expression of HAAO, reduces expression of HAAO, or inhibits HAAO activity can be selected from the group consisting of a small molecule, an antibody, an antisense nucleic acid, an aptamer, an siRNA, and combinations thereof. In some aspects, the subject can be a human. In some aspects, the subject can be diagnosed with, can be at risk for, or has Alzheimer's disease. In some aspects, the composition further g) increases expression of QPRT; h) increases expression of QPRT; i) activates QPRT; or j) degrades QA; or two or more of a) to j) in combination.

Disclosed herein are methods of reducing inflammation in a subject. In some aspects, the method comprises administering to the subject in need thereof a therapeutically effective amount of a composition that reduces production of, concentration of, or activity of quinolinic acid (QA). In some aspects, the subject can be a human. In some aspects, the subject can be diagnosed with, can be at risk for, or has Alzheimer's disease.

Disclosed herein are methods of reducing inflammation in a subject. In some aspects, the method comprises administering to the subject in need thereof a therapeutically effective amount of a composition that reduces production of, concentration of, or activity of 3-hydroxyanthranilic acid (3HAA). In some aspects, the subject can be a human. In some aspects, the subject can be diagnosed with, can be at risk for, or has Alzheimer's disease.

Disclosed herein are methods of reducing or preventing neurofibrillary tangle formation. In some aspects, the method comprises administering to a subject in need thereof a therapeutically effective amount of a composition that reduces production of, concentration of, or activity of quinolinic acid (QA). In some aspects, the subject can be a human. In some aspects, the subject can be diagnosed with, can be at risk for, or has Alzheimer's disease.

Compositions that can be used in the methods disclosed herein include but are not limited to HAAO inhibitors. In some aspects, the HAAO inhibitor can be 4CL-3HAA. 4CL-3HAA is a 3HAA analog that irreversibly binds to the HAAO enzyme. It prevents the HAAO enzyme from interacting with 3HAA and also causes it to become degraded. By preventing HAAO from converting 3HAA to QA, 3HAA can be increased and QA can be simultaneously decreased. In some aspects, the HAAO inhibitor can be 6CL-TRP. 6CL-TRP is a tryptophan analog that is converted in the body to 4CL-3HAA.

Other compositions that can be used in the methods disclosed herein include but are not limited to compositions that supplement 3HAA. In some aspects, the supplement can be 3HAA. 3HAA elevates physiological levels of 3HAA.

The compositions disclosed herein that target HAAO and/or 3HAA can be used in methods to improve or increase lifespan; improve health with age; reduce the risk or treat stroke, cardiovascular disease, Alzheimer's disease, and other neurodegenerative diseases, including Huntington's disease and Parkinson's disease.

Disclosed are methods of treating a subject, the method comprising administering a composition that can silence, reduce or inhibit the one or more genes, proteins or variants thereof “Silencing”, “reducing” or “inhibiting” in this context is a term generally used to refer to suppression, full or partial, of expression of a gene or a protein. For example, the gene (or protein) can be one or more of the genes, gene variants or proteins or protein variants described herein. For example, the degree of suppression can be to totally abolish production of the encoded gene product or a partial abolition of expression wherein some degree of expression remains. Methods of treating a subject by administering a composition that can silence, reduce or inhibit the one or more genes, proteins or variants thereof wherein said composition silences, reduces or inhibits expression of the one or more genes, proteins or variants thereof in the subject.

In some instances, silencing one or more genes, proteins or variants thereof comprises a technique well known in the art. For example, silencing can be performed by silencing transcription or by silencing translation, both of which result in a suppression of the expression of the gene. Several known techniques can be used for silencing, such as but not limited to RNAi, CRISPR, or siRNA. Silencing can comprise administering a silencing agent.

Any suitable route of administration can be used for the disclosed compositions. Suitable routes of administration can, for example, include topical, enteral, local, systemic, or parenteral. For example, administration can be epicutaneous, inhalational, enema, conjunctival, eye drops, ear drops, alveolar, nasal, intranasal, enteral, oral, intraoral, transoral, intestinal, rectal, intrarectal, transrectal, injection, infusion, intravenous, intraarterial, intramuscular, intracerebral, intraventricular, intracerebroventricular, intracardiac, subcutaneous, intraosseous, intradermal, intrathecal, intraperitoneal, intravesical, intracavernosal, intramedullar, intraocular, intracranial, transdermal, transmucosal, transnasal, inhalational, intracisternal, epidural, peridural, intravitreal, etc. The disclosed compositions can be used in and with any other therapy.

Delivery of siRNA. siRNAs, RNA that comprises or forms a double-stranded structure containing a first strand comprising a ribonucleotide sequence which corresponds to a nucleotide sequence of the target gene and a second strand comprising a ribonucleotide sequence which is complementary to the nucleotide sequence of the target gene, wherein the first and the second ribonucleotide sequences are separate complementary sequences that hybridize to each other to form said double-stranded structure, can be delivered to cells in a variety of known mechanisms.

In some aspects, siRNA can be delivered using an expression construct that encodes the siRNA. Thus, cells can be transfected with the expression construct to get the siRNA inside the cell.

In some aspects, contacting the cell or providing the cell with an RNA comprises introducing the RNA comprising a double-stranded structure into the cell using a nanoparticle carrier. Other known delivery vehicles can be used. In some aspects, the naked RNA can be delivered.

For in vivo delivery, the vehicle (RNA and associated delivery agent) can be small. For example, the vehicle can be, but is not limited to, less than 100 nm in diameter, less than 50 nm, less that 20 nm, or less than 10 nm.

In some aspects, a therapeutically effective amount of any of the compositions, including pharmaceutical compositions described herein can be administered to the subject. In some aspects, the composition can further comprise a pharmaceutically acceptable carrier; and can be administered to the subject. In some aspects, the administration of any of the compositions described herein can reduce one or more symptoms of a disease or condition, for example, Alzheimer's disease or Huntington's disease.

The pharmaceutical compositions described herein can be formulated to include a therapeutically effective amount of any the compositions disclosed herein. In some aspects, the compositions can be contained within a pharmaceutical formulation. In some aspects, the pharmaceutical formulation can be a unit dosage formulation. In some aspects, the compositions can administered on an as-needed basis.

Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to one or more side effects associated with Alzheimer's disease or Huntington's disease.

The pharmaceutical compositions described herein can be administered to the subject (e.g., a human patient) in an amount sufficient to delay, reduce, or prevent one or more symptoms of Alzheimer's disease or Huntington's disease in a subject. Accordingly, in some aspects, the patient can a human patient. In therapeutic applications, compositions are administered to a subject (e.g., a human patient) already expressing or diagnosed with one or more symptoms in an amount sufficient to at least partially improve a sign or symptom or to inhibit the progression of (and preferably arrest) the symptoms of the condition, its complications, and consequences. An amount adequate to accomplish this is defined as a therapeutically effective amount. A therapeutically effective amount of a pharmaceutical composition can be an amount that achieves a cure or reverses one or more symptoms of Alzheimer's disease or Huntington's disease, but that outcome is only one among several that can be achieved. In some aspects, the A therapeutically effective amount of a pharmaceutical composition can be an amount that increases lifespan. As noted, a therapeutically effect amount includes amounts that provide a treatment in which the onset, progression or expression of one or more of the side effects associated with Alzheimer's disease or Huntington's disease is delayed, hindered, or prevented, or the one or more symptoms associated with Alzheimer's disease or Huntington's disease is reduced, ameliorated or reversed. One or more of the symptoms can be less severe. Recovery can be accelerated in an individual who has been treated.

Amounts effective for this use can depend on the severity of the symptoms of the disease or condition and the weight and general state and health of the subject. The total effective amount of any of the compositions disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The therapeutically effective amount or dosage of the compositions used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of side effects of the synthetic cannabinoids), the age and physical characteristics of the subject and other considerations known to those of ordinary skill in the art.

Pharmaceutical Compositions

As disclosed herein, are pharmaceutical compositions, comprising any of the compositions disclosed herein and a pharmaceutical acceptable carrier. In some aspects, the compositions can be formulated for intravenous administration. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes rimonabant dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

The compositions and methods disclosed herein can be used to treat atherosclerosis, spinal cord injury or autoimmune encephalomyelitis. In some aspects, the compositions and methods are not used for treating subjects that do not have atherosclerosis, spinal cord injury or autoimmune encephalomyelitis.

Methods of Screening

Described herein are methods of screening for an agent or combination of agents effective in preventing or reducing oxidative stress; preventing one or more proteins from misfolding or aggregating; promoting proper folding of one or more proteins; increasing H₂O₂ breakdown; increasing or extending a subject's lifespan; decreasing quinolinic acid; preventing or reducing neurofibrillary tangles; preventing protein misfolding or protein aggregation; treating a disease or a condition associated with misfolding of a protein or protein aggregation or a symptom thereof; reducing amyloid-beta production; preventing oxidative stress or increasing H₂O₂ degradation; reducing inflammation; or reducing or preventing neurofibrillary tangle formation.

Specifically, described herein are methods for screening for an agent or combination of agents effective in preventing or reducing oxidative stress; preventing one or more proteins from misfolding or aggregating; promoting proper folding of one or more proteins; increasing H₂O₂ breakdown; increasing or extending a subject's lifespan; decreasing quinolinic acid; preventing or reducing neurofibrillary tangles; preventing protein misfolding or protein aggregation; treating a disease or a condition associated with misfolding of a protein or protein aggregation or a symptom thereof; reducing amyloid-beta production; preventing oxidative stress or increasing H₂O₂ degradation; reducing inflammation; and/or reducing or preventing neurofibrillary tangle formation comprising: a) determining the expression level of 3-hydroxyanthranilic acid dioxygenase (HAAO) and/or HAAO in a sample from the subject and/or determining the activity level of HAAO and/or HAAO in a sample from the subject; b) contacting the sample with the agent or combination of agents; c) determining the expression level of the HAAO and/or HAAO in the sample from the subject; and d) comparing the expression level to the expression level of HAAO or HAAO, respectively, and/or comparing the activity level to the activity level of HAAO and/or HAAO in a sample from the subject prior to contacting the sample with the agent or combination of agents to the expression level and/or activity level of HAAO or HAAO in the sample after contacting the sample with the agent or combination of agents, wherein an increase in the expression level or an increase in the activity of the HAAO and/or HAAO in the subject after contacting the sample with the agent or combination of agents indicates efficacy of the pharmaceutical agent. In some aspects, the sample from the subject can comprise or more cells.

Also described herein are methods of screening for an agent or combination of agents effective in ameliorating one or more symptoms associated with an aberrant kynurenine metabolism associated condition comprising: a) determining the expression level of HAAO or HAAO in a sample from the subject; b) contacting the sample with the agent or combination of agents; c) determining the expression level of the HAAO or HAAO in the sample; and d) comparing the expression level to the expression level of HAAO or HAAO prior to contacting the sample with the pharmaceutical agent to the expression level of the HAAO or HAAO in the sample after contacting the sample with the agent or combination of agents, wherein an increase in the expression level of the HAAO or HAAO in the sample after the contacting step indicates efficacy of the agent or combination of agents. In some aspects, the aberrant kynurenine metabolism associated condition can be, but is not limited to, diabetes, cancer, cardiovascular disease, a neurodegenerative disorder, kidney disease, or chronic inflammation. In some aspects, the sample from the subject can comprise one or more cells.

Further described herein are methods of screening for an agent or combination of agents effective in treating an aberrant kynurenine metabolism associated condition comprising: a) determining the activity of HAAO or HAAO in a sample from a subject; b) contacting the sample with the agent or combination of agents; c) determining the activity of HAAO or HAAO in the sample; and d) comparing the activity level to the activity level of HAAO or HAAO prior to contacting the sample with the agent or combination of agents, wherein an increase in the activity of HAAO or HAAO in the sample after the contacting steps indicates efficacy of the agent or combination of agents. The aberrant kynurenine metabolism associated condition can be, but is not limited to, diabetes, cancer, cardiovascular disease, a neurodegenerative disorder, kidney disease or chronic inflammation. In some aspects, the sample from the subject can be cancer cells.

Also described herein are methods of screening for an agent or combination of agents effective in treating a mitochondrial kynurenine oxidation defect comprising administering one or more agents or combination of agents to a subject, determining the expression or activity of HAAO or HAAO in the subject, and comparing those expression or activity levels to the expression or activity levels of HAAO or HAAO prior to administering the agent or combination of agents, wherein an increase in the expression or activity of HAAO or HAAO in the subject after administration indicates efficacy of the agent or combination of agents. In some aspects, the pharmaceutical agent can be an orphan drug.

Disclosed herein is a method of screening for an agent or combination of agents effective in treating an aberrant kynurenine metabolism associated condition, the method comprising (a) determining the expression level of HAAO or HAAO in a first sample from a subject; (b) administering a composition comprising the agent or combination of agents to the subject; (c) determining the expression level of HAAO or HAAO in a second sample from the subject; and (d) comparing the expression level of HAAO or HAAO obtained in step (c) to the expression level of HAAO or HAAO obtained in step (a); wherein if the expression level obtained in step (c) is greater than the expression level obtained in step (a), then the composition comprising the agent or combination of agents is effective in treating an aberrant kynurenine metabolism associated condition; wherein if the expression level obtained in step c is about equal to the expression level obtained in step (a), then the composition comprising the agent or combination of agents is not effective in treating an aberrant kynurenine metabolism associated condition; and wherein if the expression level obtained in step (c) is less than the expression level obtained in step (a), then the composition comprising the agent or combination of agents is not effective in treating an aberrant kynurenine metabolism associated condition. In some aspects, a decrease in the expression level of HAAO or HAAO obtained in step (d) when compared to the expression level of HAAO or HAAO in the normal sample can indicate that the subject has an aberrant kynurenine metabolism associated condition. In some aspects, an aberrant kynurenine metabolism associated condition can comprise cancer, cardiovascular disease, a neurodegenerative disorder, kidney disease, diabetes, or chronic inflammation.

In some aspects, a disclosed method can comprise using the agent or combination of agents identified in step (d) to treat the subject. In some aspects, an agent or combination of agents can ameliorate one or more symptoms associated with an aberrant kynurenine metabolism associated condition. In some aspects, treatment can restore HAAO or HAAO expression in the subject. In some aspects, an agent or combination of agents can be an orphan drug. In some aspects, an agent or combination of agents can be a 3HAA or TRP supplement.

Disclosed herein is a method of screening for an agent or combination of agents effective in treating an aberrant kynurenine metabolism associated condition, the method comprising (a) determining the activity level of HAAO or HAAO in a first sample from a subject; (b) administering a composition comprising the agent or combination of agents to the subject; (c) determining the activity level of HAAO or HAAO in a second sample from the subject; and (d) comparing the activity level of HAAO or HAAO obtained in step (c) to the activity level of HAAO or HAAO obtained in step (a), wherein if the activity level of obtained in step (c) is greater than the activity level obtained in step (a), then the composition comprising the agent or combination of agents is effective in treating an aberrant kynurenine metabolism associated condition; wherein if the activity level obtained in step (c) is about equal to the activity level obtained in step (a), then the composition comprising the agent or combination of agents is not effective in treating an aberrant kynurenine metabolism associated condition; and wherein if the activity level obtained in step (c) is less than the activity level obtained in step (a), then the composition comprising the agent or combination of agents is not effective in treating an aberrant kynurenine metabolism associated condition. In some aspects, a disclosed method can comprise using the agent or combination of agents identified in step (d) to treat the subject. In some aspects, an aberrant kynurenine metabolism associated condition can comprise cancer, cardiovascular disease, a neurodegenerative disorder, diabetes, kidney disease or chronic inflammation. In some aspects of a disclosed method, treatment can restore HAAO or HAAO activity in the subject. In some aspects, an agent or combination of agents can be an orphan drug. In some aspects, an agent or combination of agents can be a 3HAA or TRP supplement.

Disclosed herein is a method of screening for an agent or combination of agents effective in treating an aberrant kynurenine metabolism associated condition, the method comprising: (a) determining the amount of HAAO or HAAO in a first sample from a subject; (b) administering a composition comprising the agent or combination of agents to the subject; (c) determining the amount of HAAO or HAAO in a second sample from the subject; and (d) comparing the amount of HAAO or HAAO obtained in step (c) to the amount of HAAO or HAAO obtained in step (a), wherein if the amount of obtained in step c is greater than the activity level obtained in step a, then the composition comprising the agent or combination of agents is effective in treating an aberrant kynurenine metabolism associated condition; wherein if the amount obtained in step (c) is about equal to the amount obtained in step (a), then the composition comprising the agent or combination of agents is not effective in treating an aberrant kynurenine metabolism associated condition; and wherein if the amount obtained in step (c) is less than the activity level obtained in step (a), then the composition comprising the agent or combination of agents is not effective in treating an aberrant kynurenine metabolism associated condition. In some aspects, a disclosed method can comprise using the agent or combination of agents identified in step (d) to treat the subject. In some aspects, an aberrant kynurenine metabolism associated condition can comprise cancer, cardiovascular disease, a neurodegenerative disorder, kidney disease, chronic inflammation and diabetes. In some aspects of a disclosed method, treatment can restore HAAO or HAAO activity in the subject. In some aspects, an agent or combination of agents can be an orphan drug. In some aspects, an agent or combination of agents can be a 3HAA or TRP supplement.

Articles of Manufacture

The composition described herein can be packaged in a suitable container labeled, for example, for use as a therapy to treat or prevent synthetic cannabinoid toxicity or overdose. Accordingly, packaged products (e.g., sterile containers containing the composition described herein and packaged for storage, shipment, or sale at concentrated or ready-to-use concentrations) and kits, including at least one or more of the compositions as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compositions can be ready for administration (e.g., present in dose-appropriate units), and may include a pharmaceutically acceptable adjuvant, carrier or other diluent. Alternatively, the compositions can be provided in a concentrated form with a diluent and instructions for dilution.

### EXAMPLES

### Example 1: HAAO Inhibition as a Molecular Target for Treating Alzheimer's Disease and Huntington's Disease

Two specific mechanistic hypotheses linking 3HAA to Aβ will be tested: (1) 3HAA shifts APP production away from β-secretase toward α-secretase by degrading hydrogen peroxide (H₂O₂) and (2) 3HAA prevents Aβ from misfolding and aggregating in cell culture. A cellular model of APP processing and Aβ accumulation (BE(2)-M17 cells) in a mouse model of AD amyloid pathology (5×FAD mice) will be employed in combination with in vitro Aβ aggregation assays. Haao will be knocked out in 5×FAD mice to evaluate the impact on downstream cognitive consequences of amyloid pathology.

Huntington's disease is characterized by progressive neurodegeneration typically presenting between the ages of 30 and 50. Huntington's disease is caused by a heritable expansion of the nucleotide triplet “CAG” in the gene Huntingtin. CAG encodes a glutamine amino acid in the Huntingtin protein produced by this gene, resulting in an expanded polyglutamine region. This mutant form of Huntingtin is toxic to cells and prone to aggregation, analogous to Aβ in Alzheimer's disease. The mechanistic linking 3HAA to Huntington's pathology is less clear, but may act through similar processes to those described herein. In some aspects, 3HAA may bind Huntingtin directly and act as a small-molecule chaperone, or upregulate unfolded protein response (UPR) pathway, which can in turn upregulate expression of protein chaperones and prevent misfolding and/or aggregation of Huntingtin indirectly. These mechanisms can be evaluated in cell culture and mouse models of Huntington's disease.

Described below is the experiments and results showing the identification of HAAO and the development of a working model for a 3HAA- and QA-mediated link to AD pathology.

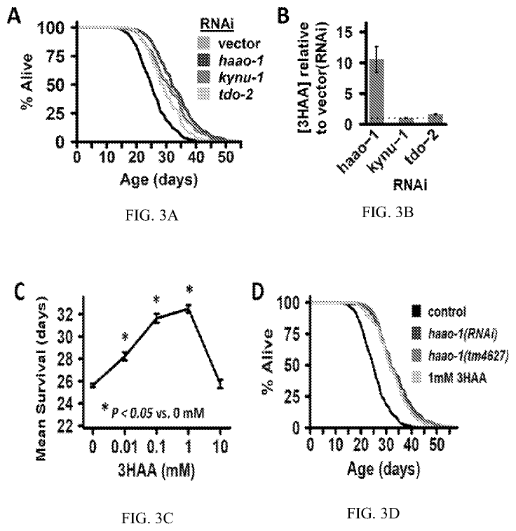

Identification of kynurenine metabolism as a regulator of survival. In a robust meta-analysis, 1,497 genes were identified that change expression with age in human whole blood (Peters M J, et al. Nat Commun 2015, 6:8570). RNAi was used to screen orthologs of these genes in C. elegans and it was discovered that knocking down kynu-1, encoding the enzyme kynureninase (KYNU; FIG. 1), extends lifespan by ˜25% (FIG. 3A) (Sutphin G L, et al. Aging Cell 2017, 16:672-82; and Sutphin G L, et al. PLOS Comput Biol 2016, 12:e1005182) Subsequently, it was found that the knockdown of two additional kynurenine pathway enzymes—tryptophan 2,3-dioxygenase (TDO/tdo-2) (van der Goot A T, et al. Proc Natl Acad Sci 2012, 109:14912-7) or HAAO/haao-1—robustly increases longevity (FIG. 3A). Initially the focus was on HAAO, knockdown of which produced the largest lifespan increase (˜30%) and improved health throughout life.

3HAA mediates lifespan extension by haao-1 knockdown. 3HAA is red in color. Worms with reduced HAAO turned visibly red with age. Using liquid chromatography-tandem mass spectrometry (LC-MS/MS), it was confirmed that these worms had elevated 3HAA (FIG. 3B). Next, it was assessed whether 3HAA may mediate the benefits of reducing haao-1. Indeed, worms supplemented with 3HAA were long-lived (FIG. 3C), mimicking haao-1 knockdown (FIG. 3D).

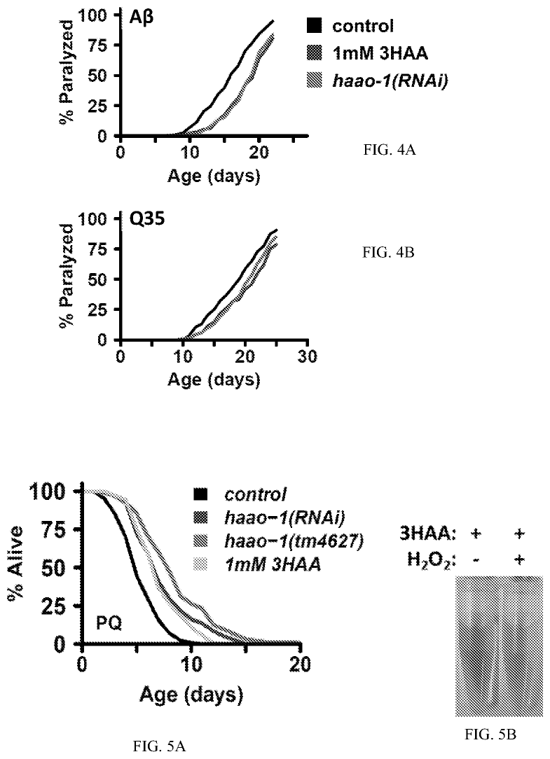

3HAA mitigates Aβ proteotoxicity. 3HAA was computationally predicted to bind Aβ and prevent misfolding, and subsequently shown to prevent Aβ aggregation in vitro (Meek A, et al. J Psychiatry Neurosci 2013, 38:269-75). It was then tested whether 3HAA would prevent Aβ toxicity in vivo. In C. elegans, transgenic expression of the aggregate-prone 42 amino acid form of Aβ (Aβ₁₋₄₂) in body wall muscle induces paralysis (Link C D. Proc Natl Acad Sci USA 1995, 92:9368-72). 3HAA supplementation or haao-1 knockdown delayed onset of paralysis in this model (FIG. 4A). 3HAA supplementation or haao-1 knockdown similarly delayed paralysis in a related model of Huntington's disease expressing an aggregate-prone polyglutamine repeat in body wall muscle (FIG. 4B). Next and as described below, 3HAA will be tested as a treatment for amyloid pathology in a cellular context more relevant to AD.

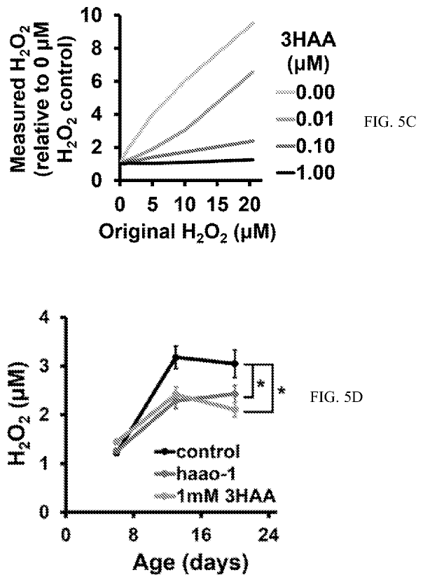

3HAA improves oxidative stress resistance and directly degrades H₂O₂. 3HAA prevents LDL oxidation in cultured human blood cells (Christen S, et al. J Clin Invest 1994, 93:2149-58; Thomas S R, et al. J Biol Chem 1996, 271:32714-21) and lipid peroxidation in the brains of rats (Leipnitz G, et al. Neurochem Int 2007, 50:83-94), which lead to evaluating whether 3HAA would improve oxidative stress resistance in C. elegans. 3HAA supplementation or haao-1 knockdown increased survival of worms exposed to paraquat, an in vivo superoxide generator (Bagley A C, et al. Proc Natl Acad Sci USA 1986, 83:3189-93; and Krall J, et al. Arch Biochem Biophys 1991, 286:311-5) (FIG. 5A). Superoxide dismutases degrade cellular superoxide to oxygen and H₂O₂. In preparing 3HAA media for oxidative stress assays, the addition of H₂O₂ substantially diminished the red coloration (FIG. 5B), suggesting a direct interaction. This interaction was interrogated in the absence of other media components and it was found that the addition of 3HAA reduced measureable H₂O₂ in vitro (FIG. 5C). Finally, 3HAA supplementation or haao-1 knockout limited endogenous H₂O₂ produced by C. elegans in vivo (FIG. 5D). In conclusion, these results show that 3HAA improves oxidative stress resistance, at least in part, by directly degrading H₂O₂, providing a mechanistic link to amyloid pathology (FIG. 2).

Published and the data described herein provide evidence for HAAO inhibition as a molecular target for treating AD. The following experiments will test the link between HAAO to AD, specifically with respect to the impact of 3HAA on Aβ production and aggregation (Example 2) and the role of QA in promoting tau hyperphosphorylation and seeding aggregation of tau and/or Aβ (Example 3). This approach combines in vitro and cellular tools to evaluate each mechanism, followed by validation in vivo using mouse models.

Mouse models. The mouse studies represent the first in vivo investigation of Haao manipulation during AD. To evaluate amyloid and tau pathology independently, separate mouse strains were selected that recapitulate AD-associated amyloid and tau pathology. For amyloid pathology, 5×FAD mice, which co-overexpress familial AD (FAD) mutant forms of human APP and presenilin 1 (PS1) in neurons (Oakley H, et al. J Neurosci Off J Soc Neurosci 2006, 26:10129-40) will be used. 5×FAD mice display rapid accumulation and aggregation of Aβ (Oakley H, et al. J Neurosci Off Soc Neurosci 2006, 26:10129-40; Ohno M, et al. Neurobiol Dis 2007, 26:134-45; Devi L, and Ohno M. Neuroscience 2015; 307:128-37), which can be reduced by intervening in APP proteolytic processing (Ohno M, et al. Neurobiol Dis 2007, 26:134-45; and Devi L, Ohno M. Neuroscience 2015, 307:128-37) or directly targeting Aβ (Wirths O, et al. J Biol Chem 2010, 285:41517-24; Wirths O, et al. J Biol Chem 2010, 285:41517-24; and Wirths O, et al. J Biol Chem 2010, 285:41517-24). For tau pathology, htau mice, which combine homozygous knockout of mouse microtubule associated protein tau (Mapt) with transgenic expression of wild-type human MAPT (Andorfer C, et al. J Neurochem 2003, 86:582-90) will be used. htau mice express six isoforms of human tau and display both tau hyperphosphorylation and accumulation of NFTs (Andorfer C, et al. J Neurochem 2003, 86:582-90). To model HAAO inhibition, 5×FAD and htau mice will be crossed to mice lacking the Haao gene (from the International Mouse Phenotyping Consortium; Dickinson M E, et al. Nature 2016, 537:508-14). The mouse studies will be conducted in 15 male and 15 female mice from each of the following groups: wild-type, Haao^{-/-}, 5×FAD, 5×FAD Haao^{-/-}, htau, htau Haao^{-/-}. A small panel of behavioral phenotyping will be conducted at 6 mo and 12 mo, including spontaneous alternation in Y-maze, novel object recognition, contextual fear conditioning, nest building, burrowing activity. These tasks were selected because 5×FAD (Oakley H, et al. J Neurosci Of J Soc Neurosci 2006, 26:10129-40; Devi L, Ohno M. Neuroscience 2015, 307:128-37; Devi L, Ohno M. Neuropsychopharmacol Off Publ Am Coll Neuropsychopharmacol 2012, 37:434-44; Park J-C, et al. BMC Complement Altern Med 2016, 16; Devi L, Ohno M. PloS One 2010, 5:e12974) and/or htau (Geiszler P C, et al. Neuroscience 2016, 329:98-111; Polydoro M, et al. J Neurosci Of Soc Neurosci 2009, 29:10741-9; Schoch K M, et al. Neuron 2016, 90:941-7; and Andorfer C, et al. J Neurosci Off J Soc Neurosci 2005, 25:5446-54). mice exhibit deficits at one or both time points. Tissues will be collected following the 12 mo testing, at which point both strains exhibit substantial pathology including neuron loss (Oakley H, et al. J Neurosci Off J Soc Neurosci 2006, 26:10129-40; Ohno M, et al. Neurobiol Dis 2007, 26:134-45; Devi L, Ohno M. Neuroscience 2015, 307:128-37; Wirths O, et al. J Biol Chem 2010, 285:41517-24; Andorfer C, et al. J Neurochem 2003, 86:582-90; Dickinson M E, et al. Nature 2016, 537:508-14; Devi L, Ohno M. Neuropsychopharmacol Off Publ Am Coll Neuropsychopharmacol 2012, 37:434-44; Park J-C, et al. BMC Complement Ahern Med 2016, 16; Devi L, Ohno M. PloS One 2010, 5:e12974; Geiszler P C, et al. Neuroscience 2016, 329:98-111; Polydoro M, et al. J Neurosci Off J Soc Neurosci 2009, 29:10741-9; Schoch K M, et al. Neuron 2016, 90:941-7; and Andorfer C, et al. J Neurosci Of f Soc Neurosci 2005, 25:5446-54)

### Example 2: Evaluate the Impact of 3HAA on Aβ Production and Aggregation

The experiments described herein evaluate the impact of 3HAA on Aβ production and aggregation. It will be determined whether supplementing cells with 3HAA can shift proteolytic processing of APP away from Aβ production, and/or prevent misfolding and aggregation of Aβ. BE(2)-M17 neuroblastoma cells, which express important enzymes and cleavage products in APP proteolytic processing (Macias M P, et al. J Neurosci Methods 2014, 223:114-22), will be used for cell culture experiments.

Determine the impact of 3HAA on APP processing. The following experiments will test whether 3HAA can shift APP processing away from β-secretase-mediated production of Aβ toward non-toxic α-secretase processing by degrading H₂O₂. A method to quantitatively detect each enzyme and proteolytic peptide produced during APP processing (see FIG. 2) using LC-MS/MS with selected reaction monitoring (SRM) will be used. In principle, SRM can be used to detect any target peptide. Using this method, APP-cleavage products will be quantitatively measured. If SRM proves infeasible, Western blots with widely published antibodies (Devi L, Ohno M. Neuroscience 2015, 307:128-37; Macias M P, et al. J Neurosci Methods 2014, 223:114-22; and Steuble M, et al. Biol Open 2012, 1:761-74), will be used. The effect of 3HAA on APP processing in BE(2)-M17 cells in the presence or absence of H₂O₂ will be measured. Elevated cellular 3HAA will be confirmed using established LC-MS/MS protocols (Sutphin G L, et al. Aging Cell 2017, 16:672-82; and Fuertig R, et al. Bioanalysis 2016, 8:1903-17). Cellular H₂O₂ levels will also be measured in the samples using commercially available kits (Abcam #ab102500). It is predicted that H₂O₂ will increase expression of APP and β-secretase while elevating APP β-secretase cleavage products relative to α-secretase cleavage products. 3HAA should reverse these changes while lowering cellular H₂O₂.

Measure the effect of 3HAA on Aβ misfolding and aggregation. Experiments will be carried out to test whether 3HAA can prevent Aβ misfolding and aggregation in living cells. BE(2)-M17 cells will be treated with synthetic Aβ₁₋₄₂ (to induce Aβ misfolding) or aggregation-incompetent scrambled-Aβ₁₋₄₂ and monitor Aβ misfolding and aggregation in cells treated with 3HAA relative to untreated controls. Misfolded Aβ will be measured using sample cell lysates to seed aggregate-free synthetic human Aβ₁₋₄₂ and aggregation will be monitored over time using thioflavin T (ThT) fluorescence. The concentration of misfolded Aβ in each sample is reflected in the timing of synthetic aggregate formation (Salvadores N, et al. Cell Rep 2014, 7:261-8). Cellular aggregates will be directly measured by ThT staining. To separate direct effects on Aβ from potential confounding effects on APP processing, observations in cells pretreated with siRNA targeting the β-secretase gene, BACE1, will be confirmed to limit endogenous Aβ production. It is predicted that 3HAA will reduce the concentration of misfolded Aβ and cellular aggregates.

Evaluate the impact of Haao knockout on amyloid pathology and cognitive deficits in 5×FAD mice. First, the cell culture observation in hippocampus and cortex samples from 5×FAD mice with or without Haao will be validated. Next, changes in APP processing, H₂O₂ concentrations, and misfolded Aβ in tissue lysate will be measured using the same methods described above for cell culture. Aβ plaque load will be further evaluated by thioflavin S (ThS) staining in histological sections. Second, the impact of Haao knockout on neuron loss and behavior will be evaluated. It is predicted that Haao knockout will protect neurons and improve performance 5×FAD mice on these tasks.

### Example 3: Evaluate the Impact of QA on Tau Phosphorylation and Aggregate Formation

The ability of QA to drive tau phosphorylation and seed aggregation of Aβ or tau will be investigated.

Determine the impact of reduced QA production on tau phosphorylation and NFTs in vivo. Because QA has already been shown to drive tau phosphorylation in cultured human neurons (Rahman A, et al. PLoS ONE 2009; 4:e6344), these experiments will focus on evaluating the effect of Haao deletion on tau pathology in htau mice. The phosphatases PP1 and PP2A, reduced expression of which has been proposed to mediate tau phosphorylation by QA (Rahman A, et al. PLoS ONE 2009, 4:e6344; Liu F, et al. Eur J Neurosci 2005, 22:1942-50; and Qian W, et al. J Alzheimers Dis JAD 2010, 19:1221-9) will also be examined. Western blots on hippocampus and cortex samples will be used to measure expression of PP1 and PP2A, and the phosphorylation state of tau at multiple sites that are elevated in htau mice (Andorfer C, et al. J Neurochem 2003, 86:582-90; Kelleher I, et al. J Neurochem 2007, 103:2256-67; and Maphis N M, et al. Front Mol Neurosci 2017, 10:69), affected by QA (Alberati-Giani D, et al. J Neurochem 1996, 66:996-1004), or targeted by PP1 and PP2A (Rahman A, et al. PLoS ONE 2009, 4:e6344; Liu F, et al. Eur J Neurosci 2005, 22:1942-50; and Qian W, et al. J Alzheimers Dis JAD 2010; 19:1221-9). The presence and co-localization of QA and NFTs will also be evaluated by immunohistochemistry using published protocols (Guillemin G J, et al. Glia 2005, 49:15-23; and Gratuze M, et al. Sci Rep 2017; 7:46359). Finally, the effect of Haao deletion on neuron loss and cognitive function will be measured in these mice. It is predicted that knocking out Haao will reduce phosphorylation and NFT load, limit or eliminate detectable QA in sectioned tissue, protect against neuron loss, and improve behavioral deficits in htau mice.

Determine whether QA can seed aggregation of either Aβ₁₋₄₂ or tau. QA self-assembles into fibrillary structures that seed aggregation of α-synuclein (Heyes M P, et al. Biochem J 1996; 320 (Pt 2):595-7). It will be tested whether these assemblies can similarly seed aggregation of Aβ and tau. First, the ability of QA assemblies to cause synthetic human Aβ₁₋₄₂ or tau to aggregate in vitro will be tested using the ThT assay described herein. Wild-type tau does not readily aggregate in a non-phosphorylated state, which may hinder the in vitro aggregation assay (Lim S, et al. Comput Struct Biotechnol J 201; 12:7-13). The assay will be performed using both wild-type tau and a more aggregate prone tau (ΔK280). In the case of a negative result the cell culture and mouse assays will be relied upon. Next, it will be determined whether QA assemblies seed aggregation in cells. For amyloid, BE(2)-M17 will be used as described herein. For tau, bimolecular fluorescence complementation (BiFC)-tagged tau expressed in HEK293 cells, which express different forms of tau are expressed, each fused to a different fragment of GFP will be used. The GFP combines and fluoresces when tau aggregates (Tak H, et al. PloS One 2013, 8:e81682). Aggregation will be monitored in both cell types following treatment with either QA assemblies or monomers. It is predicted that QA assemblies will accelerate aggregation both in vitro and in cell culture. This work will be complemented by the in vivo examination of amyloid plaques in 5×FAD mice (Example 2) and NFTs in htau mice in response to Haao deletion.

### Example 4: Molecular Mechanisms of Stress Resistance Following HAAO Inhibition

As disclosed herein, HAAO is identified as a potent molecular target to reduce oxidative and misfolded protein stress, mediated by elevated 3HAA. HAAO inhibition also limits production of the downstream, pro-oxidant metabolite QA (FIG. 1). Lowering QA may be relevant to the improved stress resistance observed during HAAO inhibition. 3HAA directly breaks down H₂O₂ (FIG. 5B) and prevents Aβ misfolding through direct binding. In human cell culture, 3HAA induces expression of the antioxidant hemeoxygenase-1 (HO-1), suggesting that indirect mechanisms may also be in play. It will be tested whether 3HAA acts, at least in part, by activating the OxSR and UPR. This experiment will be performed using available C. elegans OxSR and UPR mutants and downstream transcriptional and fluorescence reporters.

QA induces in vivo lipid peroxidation in brain tissue and drives Tau phosphorylation in vitro, a precursor to neurofibrillary tangles in Alzheimer's disease. QA also forms amyloid-like structures that seed and accelerate α-synuclein aggregation. These data suggest that QA promotes both oxidative and misfolded protein stress. QA dose response studies will be conducted to evaluate the effect on paraquat and tunicamycin resistance in C. elegans. Next, mechanistic studies will be carried out to evaluate the impact of QA on HAAO- and 3HAA-mediated stress response, OxSR and UPR activity, and pathology in protein aggregation models.

Using C. elegans will expand the inquiry of HAAO inhibition to other stressors. 3HAA can generate reactive oxygen species by reducing copper ions in vitro, suggesting that excess metal may play a role (FIG. 7). Stress response pathways in C. elegans reverse the normal benefits of 3HAA on oxidative stress. To examine this possibility, several forms of metal toxicity will also be evaluated.

HAAO is an excellent candidate for a modulator of stress response in mammals. The KP, including HAAO, is highly conserved across eukaryotes. 3HAA has been shown to activate antioxidant enzymes or prevent oxidative damage in the context of human and rodent models of cardiovascular disease, neuron cell culture, and macrophage cell culture, while QA is thought to generate reactive oxygen species and drive lipid peroxidation in rat brain. Oxidative and ER stress resistance as well as markers of oxidative damage, protein misfolding, and OxSR/UPR activation in cultured liver cells (AML12) and macrophages (RAW 264.7)—cell types from distinct tissues with active kynurenine pathway activity—subjected to HAAO inhibition or supplementation with 3HAA or QA will be examined. Primary cells from Haao^{-/-} mice will also be examined.

The mammalian KP is active in a subset of tissues and cell types and responsive to inflammatory signals. Haao^{-/-} mice will be used. Survival in response to an acute challenge with paraquat, tunicamycin, or both will be evaluated. The response to milder challenges will be further examined by evaluating OxSR and UPR activation and markers of oxidative damage and protein misfolding in several tissues.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of preventing or reducing oxidative stress in a subject, the method comprising administering to the subject a therapeutically effective amount of a composition that:
a) reduces expression of 3-hydroxyanthranilic acid dioxygenase (HAAO);
b) reduces expression of HAAO;
c) inhibits HAAO activity;
d) increases the bioavailability 3-hydroxyanthranilic acid (3HAA);
e) increases the concentration of 3-hydroxyanthranilic acid (3HAA); or
f) one or more of a) to c) in combination with d) and/or e),
thereby preventing or reducing oxidative stress in the subject.

2. The method of claim 1, wherein the administration of the composition increases break down of hydrogen peroxide.

3. A method of preventing one or more proteins from misfolding or aggregating, the method comprising administering a composition that:
a) reduces expression of 3-hydroxyanthranilic acid dioxygenase (HAAO);
b) reduces expression of HAAO;
c) inhibits HAAO activity;
d) increases the bioavailability 3-hydroxyanthranilic acid (3HAA);
e) increases the concentration of 3-hydroxyanthranilic acid (3HAA); or
f) one or more of a) to c) in combination with d) and/or e),
thereby preventing one or more proteins from misfolding or protein aggregation.

4. The method of any of claims 1 to 3, wherein the composition is administered to a subject in need thereof.

5. A method of promoting proper folding of one or more proteins, the method comprising administering a composition that:
a) reduces expression of 3-hydroxyanthranilic acid dioxygenase (HAAO);
b) reduces expression of HAAO;
c) inhibits HAAO activity;
d) increases the bioavailability 3-hydroxyanthranilic acid (3HAA);
e) increases the concentration of 3-hydroxyanthranilic acid (3HAA); or
f) one or more of a) to c) in combination with d) and/or e),
thereby promoting proper folding of one or more proteins.

6. The method of claim 5, wherein the composition is administered to a subject in need thereof.

7. A method of increasing H₂O₂ breakdown, the method comprising administering a composition that:
a) reduces expression of 3-hydroxyanthranilic acid dioxygenase (HAAO);
b) reduces expression of HAAO;
c) inhibits HAAO activity;
d) increases the bioavailability 3-hydroxyanthranilic acid (3HAA);
e) increases the concentration of 3-hydroxyanthranilic acid (3HAA); or
f) one or more of a) to c) in combination with d) and/or e),
thereby increasing H₂O₂ breakdown.

8. The method of claim 7, wherein the composition is administered to a subject in need thereof.

9. A method of increasing or extending a subject's lifespan, the method comprising administering a composition that:
a) reduces expression of 3-hydroxyanthranilic acid dioxygenase (HAAO);
b) reduces expression of HAAO;
c) inhibits HAAO activity;
d) increases the bioavailability 3-hydroxyanthranilic acid (3HAA);
e) increases the concentration of 3-hydroxyanthranilic acid (3HAA); or
f) one or more of a) to c) in combination with d) and/or e),
thereby increasing or extending the subject's lifespan.

10. The method of claim 9, wherein the composition is administered to a subject in need thereof.

11. The method of claim 9, wherein the subject is a worm.

12. A method of decreasing quinolinic acid in a subject, the method comprising administering to the subject a composition that:
a) reduces expression of 3-hydroxyanthranilic acid dioxygenase (HAAO);
b) reduces expression of HAAO; or
c) inhibits HAAO activity;
thereby decreasing quinolinic acid in the subject.

13. A method of preventing or reducing neurofibrillary tangles in a subject, the method comprising administering to the subject a composition that:
a) reduces expression of 3-hydroxyanthranilic acid dioxygenase (HAAO);
b) reduces expression of HAAO;
c) inhibits HAAO activity;
d) decrease the concentration of quinolinic acid (QA);
e) increases the bioavailability 3-hydroxyanthranilic acid (3HAA);
f) increases the concentration of 3-hydroxyanthranilic acid (3HAA); or
g) one or more of a) to c) in combination with d), e) and/or f),
thereby preventing or reducing neurofibrillary tangles in the subject.

14. A method of preventing protein misfolding or protein aggregation in a subject, the method comprising administering to the subject in need thereof a therapeutically effective amount of a composition that:
a) reduces expression of 3-hydroxyanthranilic acid dioxygenase (HAAO);
b) reduces expression of HAAO;
c) inhibits HAAO activity;
d) increases the bioavailability 3-hydroxyanthranilic acid (3HAA);
e) increases the concentration of 3-hydroxyanthranilic acid (3HAA); or
f) one or more of a) to c) in combination with d) and/or e),
thereby preventing protein misfolding or protein aggregation in the subject.

15. A method of treating a disease or a condition associated with misfolding of a protein or protein aggregation in a subject, the method comprising administering to the subject in need thereof a therapeutically effective amount of a composition that:
a) reduces expression of 3-hydroxyanthranilic acid dioxygenase (HAAO);
b) reduces expression of HAAO;
c) inhibits HAAO activity;
d) increases the bioavailability 3-hydroxyanthranilic acid (3HAA);
e) increases the concentration of 3-hydroxyanthranilic acid (3HAA); or
f) one or more of a) to c) in combination with d) and/or e),
thereby treating a disease or a condition associated with misfolding of a protein or protein aggregation in the subject.

16. The method of claim 18, wherein the disease or condition is Alzheimer's disease.

17. The method of claim 18, wherein the disease or condition is Huntington's disease.

18. A method of reducing amyloid-beta production in a subject, the method comprising administering to the subject in need thereof a therapeutically effective amount of a composition that degrades H₂O₂ or increases degradation of H₂O₂.

19. The method of claim 18, wherein the composition further:
a) reduces expression of 3-hydroxyanthranilic acid dioxygenase (HAAO);
b) reduces expression of HAAO;
c) inhibits HAAO activity;
d) increases the bioavailability 3-hydroxyanthranilic acid (3HAA);
e) increases the concentration of 3-hydroxyanthranilic acid (3HAA); or
f) increases the bioavailability and concentration of 3-hydroxyanthranilic acid (3HAA).

20. A method of preventing oxidative stress or increasing H₂O₂ degradation in a subject, the method comprising administering to the subject in need thereof a therapeutically effective amount of a composition that:
a) reduces expression of 3-hydroxyanthranilic acid dioxygenase (HAAO);
b) reduces expression of HAAO;
c) inhibits HAAO activity;
d) increases the bioavailability 3-hydroxyanthranilic acid (3HAA);
e) increases the concentration of 3-hydroxyanthranilic acid (3HAA); or
f) one or more of a) to c) in combination with d) and/or e),
thereby preventing oxidative stress or increasing H₂O₂ degradation in the subject.

21. The method of any of the proceeding claims, wherein the composition that reduces expression of HAAO, reduces expression of HAAO, or inhibits HAAO activity is a 3HAA analog, 4CL-3AA or 6CL-TRP.

22. The method of any of the proceeding claims, wherein the composition that increases or enhances the bioavailability or concentration of 3HAA is a 3HAA supplement or a 3HAA variant or analog thereof.

23. The method of any of the proceeding claims, wherein the composition that reduces expression of HAAO, reduces expression of HAAO, or inhibits HAAO activity is selected from the group consisting of a small molecule, an antibody, an antisense nucleic acid, an aptamer, an siRNA, and combinations thereof.

24. The method of any of the proceeding claims, wherein the subject is a human.

25. The method of any of the proceeding claims, wherein the subject is diagnosed with, is at risk for, or has Alzheimer's disease.

26. A method of reducing inflammation in a subject, the method comprising administering to the subject in need thereof a therapeutically effective amount of a composition that reduces production of, concentration of, or activity of quinolinic acid (QA).

27. A method of reducing inflammation in a subject, the method comprising administering to the subject in need thereof a therapeutically effective amount of a composition that reduces production of, concentration of, or activity of 3-hydroxyanthranilic acid (3HAA).

28. A method of reducing or preventing neurofibrillary tangle formation, the method comprising administering to a subject in need thereof a therapeutically effective amount of a composition that reduces production of, concentration of, or activity of quinolinic acid (QA).

29. A method of screening for an agent or combination of agents effective in preventing or reducing oxidative stress; preventing one or more proteins from misfolding or aggregating; promoting proper folding of one or more proteins; increasing H₂O₂ breakdown; increasing or extending a subject's lifespan; decreasing quinolinic acid; preventing or reducing neurofibrillary tangles; preventing protein misfolding or protein aggregation; treating a disease or a condition associated with misfolding of a protein or protein aggregation or a symptom thereof; reducing amyloid-beta production; preventing oxidative stress or increasing H₂O₂ degradation; reducing inflammation; and/or reducing or preventing neurofibrillary tangle formation comprising:
a) determining the expression level of 3-hydroxyanthranilic acid dioxygenase (HAAO) and/or HAAO in a sample from the subject;
b) contacting the sample with the agent, the agent or combination of agents;
c) determining the expression level of the HAAO and/or HAAO in the sample from the subject; and
d) comparing the expression level to the expression level of HAAO or HAAO, respectively in a sample from the subject prior to contacting the sample with the agent, the agent or combination of agents to the expression level of HAAO or HAAO in the sample after contacting the sample with the agent, the agent or combination of agents,
wherein an increase in the expression level or an increase in the activity of the HAAO and/or HAAO in the subject after contacting the sample with the agent, the agent or combination of agents indicates efficacy of the agent, the agent or combination of agents.

30. A method of screening for an agent, the agent or combination of agents effective in preventing or reducing oxidative stress; preventing one or more proteins from misfolding or aggregating; promoting proper folding of one or more proteins; increasing H₂O₂ breakdown; increasing or extending a subject's lifespan; decreasing quinolinic acid; preventing or reducing neurofibrillary tangles; preventing protein misfolding or protein aggregation; treating a disease or a condition associated with misfolding of a protein or protein aggregation or a symptom thereof; reducing amyloid-beta production; preventing oxidative stress or increasing H₂O₂ degradation; reducing inflammation; and/or reducing or preventing neurofibrillary tangle formation comprising:
a) determining the activity level of HAAO and/or HAAO in a sample from the subject;
b) contacting the sample with the agent, the agent or combination of agents;
c) determining the activity level of the HAAO and/or HAAO in the sample from the subject; and
d) comparing the activity level to the activity level of HAAO and/or HAAO in a sample from the subject prior to contacting the sample with the agent, the agent or combination of agents to the activity level of HAAO or HAAO in the sample after contacting the sample with the agent, the agent or combination of agents, wherein an increase in the activity of the HAAO and/or HAAO in the subject after contacting the sample with the agent, the agent or combination of agents indicates efficacy of the agent, the agent or combination of agents.

31. A method of screening for an agent or combination of agents effective in ameliorating one or more symptoms associated with an aberrant kynurenine metabolism associated condition comprising:
a) determining the expression level of HAAO and/or HAAO in a sample;
b) contacting the agent, the agent or combination of agents with the sample;
c) determining the expression level of the HAAO and/or HAAO in the sample; and
d) comparing the expression level to the expression level of HAAO and/or HAAO prior to contacting the sample with the agent, the compound or the combination thereof to the expression level of the HAAO and/or HAAO in the sample after contacting the sample the sample with the agent, the agent or combination of agents, wherein an increase in the expression level of the HAAO and/or HAAO in the sample after administration indicates efficacy of the agent, the agent or combination of agents.

32. A method of screening for an agent or combination of agents effective in treating an aberrant kynurenine metabolism associated condition comprising:
a) determining the activity of HAAO and/or HAAO in a sample;
b) contacting the sample with the agent or combination of agents;
c) determining the activity of HAAO and/or HAAO in the sample from the subject; and
d) comparing the activity level to the activity level of HAAO and/or HAAO prior to contacting the sample with the agent, the agent or combination of agents, wherein an increase in the activity of HAAO and/or HAAO in the subject after administration indicates efficacy of the agent or combination of agents.

33. The method of any of claims 31-32, wherein the aberrant kynurenine metabolism associated condition is cancer, a neurodegenerative disorder, diabetes, kidney disease, chronic inflammation, or cardiovascular disease.
